(19) United States
(12) Patent Application Publication
(10) Pub. No.: US 2008/0200422 A1
(43) Pub. Date: Aug. 21, 2008

(54) Methods for reduction of adipose tissue mass

(76) Inventors: Douglas R. Cavener, State College, PA (US); Feifan Guo, State College, PA (US)

Correspondence Address: MCDERMOTT WILL & EMERY LLP, 600 13TH STREET, N.W., WASHINGTON, DC, 20005-3096, US

(21) Appl. No.: 12/004,518

(22) Filed: Dec. 21, 2007

Related U.S. Application Data
(60) Provisional application No. 60/879,357, filed on Jan. 9, 2007.
(60) Provisional application No. 60/880,009, filed on Jan. 12, 2007.

(51) Int. Cl. A61K 31/70; A61K 31/196; A61K 31/4164; C12Q 1/68; A23K 1/18; A61K 31/351; A61K 31/135; A61P 3/06

(52) U.S. Cl. 514/46; 514/561; 514/400; 514/460; 514/649; 435/6; 426/2

(57) ABSTRACT

The present disclosure relates to diets, compounds, e.g., GCN2 agonists and methods that selectively reduce adipose tissue mass relative to the mass of other tissue types and increase insulin sensitivity. Another aspect of the disclosure relates to methods for identifying GCN2 agonists that will decrease the activity and/or expression of one or more of adipogenic genes thereby selectively inhibiting the cellular mechanisms that lead to fat production. Such agonists have the effect of reducing adipose tissue mass in an individual.

### CROSS-REFERENCE TO RELATED APPLICATION

This application claims the benefit of U.S. Provisional Application U.S. Ser. No. 60/879,357, which was filed on Jan. 9, 2007, and U.S. Provisional Application U.S. Ser. No. 60/880,009, which was filed on Jan. 12, 2007, both applications are hereby incorporated by reference in their entirety.

### FIELD OF THE DISCLOSURE

The present disclosure relates to methods of, and identifying compounds for, reduction of excess adipose tissue mass in humans or animals.

### BACKGROUND OF THE DISCLOSURE

Obesity is a condition in which the natural energy reserve, stored in the fatty tissue of humans and other mammals, is increased to a point where it is associated with certain health conditions or increased mortality. Obesity is both an individual clinical condition and is increasingly viewed as a serious public health problem. Excessive body weight has been shown to predispose subjects to various diseases, particularly cardiovascular diseases, diabetes mellitus type 2, sleep apnea, and osteoarthritis.

The combination of an excessive nutrient intake and a sedentary lifestyle has been identified as the main cause for the rapid acceleration of obesity in Western society in the second half of the 20th century. The prevalence of overweight and obesity in the United States in particular makes obesity a leading public health problem. The United States has the highest rates of obesity in the developed world. From 1980 to 2002, obesity has doubled in adults and overweight prevalence has tripled in children and adolescents. From 2003-2004, 17.1% of children and adolescents aged 2 to 19 years were overweight and 32.2% of adults aged 20 years or older were obese. The prevalence in the United States continues to rise.

Studies have shown that a loss of as little as 5% of body mass can create large health benefits. However, even this can be difficult to achieve and often requires medical intervention with anti-obesity drugs. Anti-obesity drugs are intended to alter one of the fundamental processes of the human body. Such drugs are medically prescribed only in cases of morbid obesity, where weight loss is life-saving. However, anti-obesity drugs often have severe and often life-threatening side effects, e.g., Fen-phen. These side effects are often associated with their mechanism of action. In general, stimulants carry a risk of high blood pressure, faster heart rate, palpitations, closed-angle glaucoma, drug addiction, restlessness, agitation, and insomnia.

Another drug, Orlistat, blocks absorption of dietary fats, and as a result may cause oily spotting bowel movements, oily stools, stomach pain, and flatulence. A similar medication, designed for patients with Type 2 diabetes, is Acarbose which partially blocks absorption of carbohydrates in the small intestine, and produces similar side effects including stomach pain, and flatulence. As such, reliable dieting agents remain elusive.

Amino acids play central roles both as building blocks of proteins and as intermediates in metabolism. The 20 amino acids that are found within proteins convey a vast array of chemical versatility. An essential amino acid or indispensable amino acid is an amino acid that cannot be synthesized de novo by an organism, and therefore must be supplied in the diet. Nine amino acids are generally regarded as essential for humans. They are: isoleucine, leucine, lysine, threonine, tryptophan, methionine, histidine, valine and phenylalanine.

Net protein utilization is profoundly affected by the limiting amino acid content (the essential amino acid found in the smallest quantity in the foodstuff), and somewhat affected by salvage of essential amino acids in the body. However, organisms have evolved a repertoire of adaptations to cope with episodes of malnutrition and starvation. To maintain vital functions during prolonged malnutrition proteins, carbohydrates, lipids and other biomolecules are mobilized from skeletal muscle, adipose tissues, and specific internal organs including the liver (see, e.g., Anthony et al., 2004, J Biol Chem 279, 36553-36561; Heard et al., 1977, Br J Nutr 37, 1-21; Munro, 1975, Infusionsther Klin Ernahr 2, 112-117; Anthony et al., Am J Physiol Endocrinol Metab, 281: E430-E439, 2001).

Amino acid deprivation in mammals is particularly problematic given the existence of the nine essential amino acids that can not be synthesized de novo. Consequently some proteins must be degraded to redeploy essential amino acids for synthesis of proteins vital for survival. Recycling of non-essential proteins in skeletal muscle and some of the internal organs such as liver, results in overall reduction in body weight (see, e.g., Adibi, 1976, Metabolism 25, 1287-1302; Kerr et al., 1978, Metabolism 27, 411-435).

Adaptive changes to amino acid deprivation have been extensively studied in yeast, where they have been shown to be regulated by the General Control Nonderepressible 2 (GCN2) kinase. GCN2 is activated by uncharged tRNA-dependent mechanisms (Hinnebusch, 1994, Semin Cell Biol 5, 417-426; Wek et al., 1995, Mol Cell Biol 15, 4497-4506). Activated GCN2 kinase phosphorylates the eukaryotic initiation factor 2-alpha (eIF2α), and results in de-repression of the translation of the transcription factor GCN4. Elevated levels of GCN4 stimulate the expression of hundreds of genes including enzymes required to synthesize all twenty major amino acids (see, e.g., Hinnebusch, 1997, J Biol Chem 272, 21661-21664; Mueller and Hinnebusch, 1986, Cell 45, 201-207).

Mice lacking GCN2 fail to alter the phosphorylation of this eIF2 initiation. Zhang et al., Mol Cell Biol. 2002 October; 22(19):6681-8, described how wild-type and GCN2 deficient mice were provided a nutritionally complete diet or a diet devoid of leucine or glycine. GCN2 deficient mice are viable, fertile, and exhibit no obvious phenotypic abnormalities under standard growth conditions. However, prenatal and neonatal mortalities are significantly increased in GCN2 deficient mice whose mothers were reared on leucine-, tryptophan-, or glycine-deficient diets during gestation. Leucine deprivation produced the most pronounced effect, with a 63% reduction in the expected number of viable neonatal mice.

Anthony et al., J Biol. Chem. 2004 Aug. 27; 279(35):36553-61, described how in wild-type mice, dietary leucine restriction resulted in loss of body weight and liver mass. In contrast, a significant proportion of GCN2 deficient mice lost skeletal muscle while liver protein expression went on unabated. The GCN2 deficient mice also died within 6 days on the leucine-deficient diet. The weight loss observed in the wild type mice was interpreted as a result of global repression of protein synthesis losing small amounts of muscle and larger amounts of liver mass. The GCN2 deficient mice were unable to down regulate translation—particularly in the liver—and as such, showed dramatic skeletal muscle loss while liver volume remained constant. The article ultimately concluded that the loss of GCN2 eIF2 kinase activity shifts the normal maintenance of protein mass away from skeletal muscle to provide substrate for continued hepatic translation. These results demonstrated that GCN2 is a sensor of amino acid deprivation that triggers a repression of global protein synthesis while simultaneously negatively acting on specific proteins associated with translation of specific classes of mRNA, i.e., 4E-BP1 and S6K1. A role for GCN2 in lipid synthesis was not previously contemplated or suggested.

Notwithstanding the state of the art's general wisdom regarding the perceived deleterious effects of a deficiency of essential amino acids, diets reduced in essential amino acids have been used in the past for various purposes. For example, U.S. Pat. No. 4,760,090 discloses feeding livestock ketoisocaproate (KIC) with a diet of limited leucine content for enhancement of growth and feed efficiency. The disclosure, states that for enhancement of growth and feed efficiency, the amount of leucine in the protein of the diet should be limited to not over 12% of the total protein being consumed, and preferably to an amount of leucine less than 10% of the total protein. Chawla et al., American Journal of Clinical Nutrition, 28; September 1975, pp. 947-949 also found that when leucine is withdrawn from the diet of a growing rat, food intake declines and the animal loses weight. However, the results observed in these studies were interpreted as a result of appetite loss and/or global repression of protein synthesis.

Diets minimizing the intake of essential amino acids have also been used therapeutically to treat particular disease. For example, familial leucine-sensitive hypoglycemia of infancy is a condition in which symptomatic hypoglycemia was provoked by protein meals in general or leucine in particular. Further, isovaleric acidemia is another rare genetic disorder in which the body is unable to process certain proteins properly. People with this disorder have inadequate levels of an enzyme that helps break down the amino acid leucine and therefore, rely on minimizing the intake of leucine in the diet. Such patients take leucine-free medical food supplements such as for example, XLeu MAXAMUM® distributed by Nutricia North America, Inc (Gaithersburg, Md.). However, it is not believed that such supplements have heretofore been thought to be suitable for treating obesity or bringing about weight loss. On the contrary, high levels of leucine are thought to promote weight loss because leucine works with insulin to stimulate protein synthesis in muscle.

There remains a general need for safe and effective dietary supplements and drugs that result in the preferential ablation of adipose tissue.

All scientific documents and references cited herein are incorporated by reference in their entirety for all purposes including: Bodenlenz et al., (2005). Am J Physiol Endocrinol Metab 289, E296-300; Guo et al., (2007) Cell Metab 5, 103-14; Hao et al., (2005), Science 307, 1776-8; Harding et al., (2003), Mol Cell 11, 619-33; Hinnebusch (1994), Semin Cell Biol 5, 417-26; Hinnebusch (1997). J Biol Chem 272, 21661-4; Maurin et al., (2005). Cell Metab 1, 273-7; Mendoza-Nunez et al., (2002) Obes Res 10, 253-9; Wek et al., (1995). Mol Cell Biol 15, 4497-506; Zhang et al. (2002). Mol Cell Biol 22, 6681-8.

### SUMMARY OF THE DISCLOSURE

It was also discovered that when fed a diet deficient in an essential amino acid, mice rapidly and preferentially lose fat from adipose tissue. It was also discovered that when fed a diet deficient in an essential amino acid, GCN2-deficient mice fail to repress fatty acid synthesis and instead increase it. In fact, GCN2-deficient mice fed a diet deficient in an essential amino acid had severe liver steatosis. In addition, the loss of abdominal fat is ablated in GCN2-deficient mice. It was discovered that the GCN2-dependent suppression of hepatic lipogenesis is mediated by repression of the SREBP-1c, a key transcriptional regulator of lipogenesis

In summary, a diet deficient in an essential amino acid causes three dramatic changes: 1) a cessation of fatty acid synthesis in the liver; 2) loss of fat from adipose tissue; and 3) increased insulin sensitivity. These changes in fat metabolism are the hallmarks of a starvation response. Because the diet deficient in an essential amino acid diet, is isocaloric and contains normal levels of carbohydrates and fats sufficient to continue fatty acid homeostasis, this starvation-like response was completely unexpected.

Without wishing to be bound to any particular mechanism of action, it is proposed that essential amino acid deficiency tricks the underlying regulatory system into acting as if starvation is occurring, and GCN2 plays a dominant role in this regulation. GCN2 not only regulates amino acid metabolism, but also functions to regulate fatty acid metabolism during nutrient deprivation. From a global metabolic perspective it appears that a limited number of nutrient sensors, e.g. for glucose and amino acids, are used by the body to regulate the metabolism of various nutrients deficient during general nutrient deprivation.

One aspect of the present disclosure relates to a method of preferentially reducing adipose tissue mass in an animal comprising: providing the animal with a diet substantially deficient in an essential amino acid for a period of time. The reduction in adipose tissue mass can be observed by measuring a reduction in the adipose tissue mass index. In one embodiment of the disclosure, the animal is a human. In another embodiment, the adipose tissue mass index is measured using a technique selected from the group consisting of bioelectrical impedance analysis, dual energy X-ray absorptiometry and determination of a body mass index (BMI). In a further embodiment, the essential amino acid is leucine. In still another embodiment, the period of time is less than about 1 year, less than about 6 months, less than about 3 months, less than about 1 month, less than about 2 weeks, or less than about 1 week or any period therebetween.

Another aspect of the disclosure relates to a method of reducing adipose tissue mass index in an animal comprising: providing the animal with a diet comprising a GCN2 agonist for a period of time; and, optionally, observing a reduction in adipose tissue mass index. In one embodiment, the animal is a human. In yet another embodiment, the GCN2 agonist is selected from the group consisting of leucinol, histindol, and threoninol. In still another embodiment, the GCN2 agonist is a tRNA aminoacylation inhibitor. In still another embodiment, the tRNA aminoacylation is selected from the group consisting of pseudomonic acid, SB-203207, SB-219383, indolmycin, capsaicin and ascamycin, an aminoalkyl adenylate and an aminoacylsulfamoyl adenosine. In still another embodiment, the period of time is less than about 1 year, less than about 6 months, less than about 3 months, less than about 1 month, less than about 2 weeks, or less than about 1 week or any period therebetween.

Another aspect of the disclosure relates to a method of identifying a GCN2 agonist comprising, growing a test cell with a GCN2 deficiency in an environment substantially free of an essential amino acid, growing a control cell with a GCN2 deficiency in the same environment; contacting the test cell a compound suspected of being a GCN2 agonist; identifying a GCN2 agonist where the test cell expresses a lower level of a target gene than the control cell. In one embodiment, the GCN2 deficiency is the result of a mutation in the cell's GCN2 gene. In another embodiment, the GCN2 deficiency is the result of a GCN2 target siRNA. In yet a further embodiment, the essential amino acid is leucine. In still another embodiment, the expression is measured by determining target gene mRNA levels. In still another embodiment, the expression level is determined by measuring target gene protein product levels. In yet another embodiment, the target gene is an adipogenic gene. In a further embodiment, the target gene comprises the promoter region of an adipogenic region operably linked to a reporter nucleic acid. In yet a further embodiment, the adipogenic gene is selected from the group consisting of fatty acid synthase (FAS), ATP-citrate lyase (ACL), glucose-6-phosphate dehydrogenase (G6PD), malic enzyme (ME), SREBP1c, S1P, PPAR-gamma, fatty aci-CoA oxidase (ACO), long and medium chain acyl-CoA dehydrogenase (LCAD and MCAD), fatty acid binding protein (FABP), fatty acid translocase (CD36), fatty acid transport protein (FATP), and lipoprotein lipase (LPL), alipoproteins B (ApoB).

Another aspect of the invention relates to a method of increasing insulin sensitivity in an animal in need thereof comprising, providing the animal with a diet substantially deficient in an essential amino acid for a period of time. In one embodiment, the animal is a human. In another embodiment, the method further comprises observing an increase in insulin sensitivity by measuring glucose and insulin levels in the animal. In a further embodiment, the increase in insulin sensitivity is measured using a quantitative insulin sensitivity check index (QUICKI) or a glucose tolerance test (GTT). In yet a further embodiment, the essential amino acid is leucine. In still another embodiment, the period of time is less than about 1 year, less than about 6 months, less than about 3 months, less than about 1 month, less than about 2 weeks, or less than about 1 week or any period therebetween.

Another aspect of the invention relates to a method of increasing insulin sensitivity in an animal in need thereof comprising providing the animal with a diet comprising a GCN2 agonist for a period of time; and optionally, observing an increase in insulin sensitivity. In one embodiment, the animal is a human. In another embodiment, the GCN2 agonist is selected from the group consisting of leucinol, histindol, and threoninol. In a further embodiment, the GCN2 agonist is a tRNA aminoacylation inhibitor. In another embodiment, the inhibitor of tRNA aminoacylation is selected from the group consisting of pseudomonic acid, SB-203207, SB-219383, indolmycin, capsaicin and ascamycin, an aminoalkyl adenylate and an aminoacylsulfamoyl adenosine. In yet a further embodiment, the essential amino acid is leucine In still another embodiment, the period of time is less than about 1 year, less than about 6 months, less than about 3 months, less than about 1 month, less than about 2 weeks, or less than about 1 week or any period therebetween.

Additional advantages of the present disclosure will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail to avoid unnecessarily obscure the present disclosure. Accordingly, the description is to be regarded as illustrative in nature, and not as restrictive.

### DESCRIPTION OF THE DRAWINGS

Table 1. Phenotypic differences between wild-type (+/+) and GCN2 knockout (−/−) mice on control and leucine-deficient diet. 2-3 months old mice were either maintained on nutritionally complete amino acid diet (control diet) or diet devoid of leucine [(−) leu diet] for 17 or 7 days. Numbers and sex of mice used: for 17 days: n=5 (3M, 2F) per treatment per genotype; for 7 days: control diet: n=6 (3M, 3F) per genotype, and (−) leu diet: n=8 (5M, 3F) per genotype. Each value represents the mean ±SEM. Total food intake refers to the total amount of control or leucine-deficient diet consumed during 17 or 7 days. *Effect of (−) leu diet versus control diet group compared within the same strains of mice (two-tailed student t-test; p<0.01.). For all parameters examined, no significant differences were observed between the two GCN2 stains fed on control diet or in the response to (−) leu diet according to gender.

Table 2. Sample menus for a low leucine diet.

Table 3. Nutrient break down for a low leucine diet.

FIG. 1. Leucine-deprived GCN2^{-/-} mice exhibit severe liver steatosis. Liver histology for wild-type (+/+) and GCN2 KO (−/−) mice. Mice were fed either control diet or leucine-deficient diet for 7 days. Liver tissue sections from control and (−) leu diet group animals were stained with oil red O, magnification 40× (A) or hematoxylin and eosin, magnification 20× (B). Lipid deposits are detected as red-stained areas in A and white empty areas in B. Shown are representative of several animals for each group.

FIG. 2. Lipogenic genes are not repressed in livers of leucine-deprived GCN2^{-/-} mice, Expression of lipogenie genes in the livers of GCN2 KO (−/−) and wild-type (+/+) mice fed either control or leucine-deprived diets for seven days. A. Expression of FAS, SCD, ACL, G6PD, ME and ACC1 mRNAs. B. FAS protein (left: western blot; right: FAS protein relative to tubulin and normalized to control diet group within same strains of mice). C. FAS enzyme activity. Data are mean ±SEM of at least two independent real-time PCR experiments per animal (A) or western blot (B) with mice of each diet treatment for each experiments [WT: control diet (n=6); (−) leu diet (n=8); KO: control diet (n=6); (−) leu diet n=8)]. *Effect of (−) leu diet versus control diet group compared within the same strains of mice (two-tailed student t-test; p<0.0001.).

FIG. 3. GCN2-dependent regulation of SREBP-1c and PPARγ. Expression of Lipogenic regulatory factors in the livers of GCN2 KO (−/−) and wild-type (+/+) mice fed either control or leucine-deprived diets for seven days. A. Expression of SREBP-1c, SCAP, SIP, Insig 1 and Insig 2a mRNA. B. SREBP-1c precursor protein (top: western blot; bottom: SREBP-1c protein relative to NS protein and normalized to control group within same strains of mice). C. SREBP-1c nuclear mature form of SREBP-1c (top: western blot; bottom: SREBP-1c protein relative to tubulin and normalized to control group within same strain of mice). D. Expression of PPARγ mRNA. E. PPARγ protein from nuclear extraction (top: western blot; bottom: PPARγ protein relative to tubulin and normalized to control group within same strain of mice). Data are mean ±SEM of at least two independent real-time PCR experiments (A and D) or western blot (B, C and E) with mice of each diet treatment for each experiments [WT: control diet (n=6); (−) leu diet (n=8); KO: control diet (n=6); (−) leu diet n=8)]. *Effect of (−) leu diet versus control diet within the same strains of mice (two-tailed student t-test; p<0.0001.).

FIG. 4. β-oxidation and fatty acid transport genes are upregulated in livers of GCN2^{-/-} mice fed a leucine-deprived diet. Expression of β-oxidation and fatty acid transport genes in the livers of GCN2 KO (−/−) and wild-type (+/+) mice fed with leucine-deficient diet for seven days compared with control diet. A. PPARα, ACO, LCAD and MCAD mRNA. B. FABP, cd36, FATP and LPL mRNA. C Apo B and E mRNA. Data are mean ±SEM of at least two independent real-time PCR experiments with mice of each diet treatment for each experiment. *Effect of (−) leu diet versus control diet group compared within the same strains of mice (two-tailed student t-test; p<0.0001.). [WT: control diet (n=6); (−) leu diet (n=8); KO: control diet (n=6); (−) leu diet n=8)1.

FIG. 5. Leucine deprivation induced-liver steatosis in GCN2^{-/-} mice is prevented by inhibition of FAS expression. GCN2 KO (−/−) mice were fed a leucine-deprived diets for seven days and were given C75 at a dose of 20 mg/Kg body weight in 200 μl RPMI or only RPMI every other day by IP injection during this period. A. Liver histology for GCN2 KO mice. Sections from animals were stained with oil red O, magnification 40×. Lipid deposits are detected as red-stained areas. B and C. Expression of FAS mRNA (B) and FAS protein (C, top: western blot; bottom: FAS protein relative to tubulin and normalized to control diet group within same strains of mice). *Effect of with C75 treatment fed on (−) leu diet versus without C75 treatment fed on (−) leu diet or control diet in GCN2 KO mice (two-tailed student t-test; p<0.0001.). [KO: control diet (n=6); (−) leu diet (n=5); (−) leu diet+C75 (n=3)]. D and E. Expression of PPARα and ACO mRNA. *Effect of with C75 treatment versus without C75 treatment on either (−) leu diet (*a: p<0.001) or control diet group (*b: p<0.001) in GCN2 KO mice (two-tailed student t-test; p<0.0001.). [KO: control diet (n=6); (−) leu diet (n=5); (−) leu diet+C75 (n=3)].

FIG. 6. Phosphorylation of eIF2α and regulation of amino acid biosynthesis genes are GCN2-dependent in the liver under leucine deprivation. GCN2 KO (−/−) and wild-type mice (+/+) were fed control or leucine-deficient diet for seven days. A. phosphorylation of eIF2α (top: western blot; bottom: phospho-eIF2α relative to eIF2α and normalized to control diet group within same strains of mice). B. ASNS mRNA. C. CIEBP,8 mRNA. D. C/EBPβ protein from nuclear extraction (top: western blot; bottom: C/EBPβ protein relative to tubulin and normalized to control diet group within same strains of mice). *Effect of (−) leu diet versus control diet within the same strains of mice (two-tailed student t-test; p<0.0001.). [WT: control diet (n=6); (−) leu diet (n=8); KO: control diet (n=6); (−) leu diet n=8)]. E. Model of GCN2-dependent regulation of lipid metabolism in liver. Leucine deprivation results in activation of GCN2 and phosphorylation of cIF2α. This is followed by the repression of SREBP-1c and its downstream lipogenic genes and in parallel activation of ATF4 and C/EBPβ, as well as their downstream amino acid regulatory genes.

FIG. 7. GCN2^{-/-} mice have normal glucose tolerance response. Mice were fasted overnight for 14 h followed by i.p. injection of glucose (1 mg/g body weight). Blood samples were obtained from tail veins at 0, 5, 10, 15, 30, 60, and 120 minutes after injection, and the glucose levels were assessed by using a glucometer (One Touch, Lifescan).

FIG. 8. ChREBP and L-PK mRNA are not altered by leucine deprivation in GCN2^{-/-} mice. Expression of ChREBP and L-PK mRNA in the livers of GCN2 KO (−/−) and wild type (+/+) mice fed either control or leucine-deprived diets for seven days. Data are mean ±SEM of at least 2 independent real time PCR experiments [WT: control diet (n+6); (−) leu diet (n=6); KO: control diet (n=6); (−) leu diet (n=6)].

FIG. 9. Leucine-deprived ATF4^{-/-} mice do not develop fatty liver. A. Liver histology for a wild type (ATF4+/+) and ATF4 KO (−/−) mice. Mice were fed either control diet or leucine deficient diet for 7 days. Liver tissue sections from control and (−) leu diet group animals were stained with Oil red O, magnification 40×. Lipid deposits are detected as red-stained areas. Shown are representatives of several animals for each group. B. FAS (left) and ASNS mRNA (right). Data are mean ±SEM of at least 2 independent real-time PCR experiments with mice of each diet treatment for each experiment [WT: control diet (n=6); (−) leu diet (n=6); KO: control diet (n=6); (−) leu diet (n=6)].

FIG. 10. Leucine-deprived mice exhibit increased glucose clearance in glucose tolerance test. Mice were previously fed either a complete synthetic diet or the same diet deficient for leucine for seven days. Mice were injected with a bolus of glucose and serum glucose levels were subsequently measured after glucose injection (time zero). Mice previously deprived of leucine for seven days exhibit a faster clearance of glucose than mice fed a normal, complete diet. Combined with the observation that leucine-deprived mice have lower serum insulin levels with normal glucose levels, the results of the glucose tolerance test indicate that leucine-deprived mice have increased insulin sensitivity.

### DETAILED DESCRIPTION OF THE DISCLOSURE

During episodes of extended fasting, lipid synthesis in the liver is down-regulated and lipolysis is upregulated in adipose tissue in concert with an overall shift in the balance of anabolic and catabolic metabolism (See, e.g., Finn and Dice, 2006, Nutrition 22, 830-844). Being deprived of only a single essential amino acid would, however, seem not to impact or be relevant to lipid metabolism. Surprisingly, it was discovered that lipid synthesis is down-regulated in the liver, and adipose tissue mass is rapidly reduced in wild-type mice fed a leucine-deficient diet for several days. Moreover it was discovered that that wild-type mice in these leucine-deprived dietary experiments had increased insulin sensitivity.

It was further recognized, through experimentation and investigation, that the limitation of even a single essential amino acid triggers a global nutrient deprivation response that includes the modulation of lipid synthesis. As essential amino acid limitation in nature occurs during general nutrient deprivation, it was determined that detection of essential amino acid limitation acts to mobilize a global adaptive metabolic response.

A deficiency of an essential amino acid is sensed by the General Control Nonderepressible 2 (GCN2) kinase. It was discovered that GCN2 is directly involved in repressing lipid synthesis during amino acid deprivation. This was shown examining the response of GCN2^{+/+} and GCN2^{-/-} mice to deprivation of the essential amino acid leucine. It was found that that lipid synthesis was repressed in the livers of GCN2^{+/+} mice during prolonged leucine deprivation whereas lipid synthesis continued unabated in GCN2^{-/-} mice, resulting in severe liver steatosis. As such, the expression of lipogenic genes and the activity of fatty acid synthase (FAS) in the liver are repressed and lipid stores in adipose tissue are mobilized in mammals upon leucine deprivation. Failure to down-regulate lipid synthesis was found to be due to persistent expression of sterol regulatory element-binding protein SREBP-1c, its downstream transcriptional targets underlying fatty acid and triglyceride synthesis. Thus, a novel function of GCN2 in regulating lipid metabolism during leucine deprivation in addition to regulating amino acid metabolism was implicated.

The term “adipose tissue” as used herein refers loose connective tissue composed of adipocytes. Its main role is to store energy in the form of fat, although it also cushions and insulates the body. Obesity in humans and most animals is not dependent on the amount of body weight, but on the amount of body fat—specifically adipose tissue. Two types of adipose tissue exist: white adipose tissue (WAT) and brown adipose tissue (BAT). Adipose tissue is located beneath the skin and is also found around internal organs. Adipose tissue is found in specific locations, which are referred to as ‘adipose depots’. Adipose tissue contains several cell types, with the highest percentage of cells being adipocytes, which contain fat droplets. Other cell types include fibroblasts, macrophages and endothelial cells. Adipose tissue contains many small blood vessels. In the integumentary system, which includes the skin, it accumulates in the deepest level, the subcutaneous layer, providing insulation from heat and cold. Around organs, it provides protective padding. However, its main function is to be a reserve of lipids, which can be burned to meet the energy needs of the body.

The present disclosure relates to diets, compounds and methods that selectively reduces adipose tissue mass relative to the mass of other tissue types. The reduction in adipose tissue mass can be the result either of the shrinkage of adipocytes themselves or the actual reduction in the number of adipose cells in the tissue through apoptosis, for example.

An “adipose tissue mass index” as referred to herein involves measuring the mass of adipose tissue relative to total animal mass. Since fat tissue has a lower density than muscles and bones, it is possible to estimate a body's fat content. Preferably, the index is measured before, during and after a particular dietary or therapeutic regimen in order to gauge the effectiveness of the dietary or therapeutic regimen in preferentially reducing adipose tissue mass.

Most preferably, this is be done by determining a body fat percentage, for example. Body fat percentage is an estimate of the fraction of the total body mass that is adipose tissue (or also referred to as ‘fat mass’), as opposed to lean body mass (muscle, bone, organ tissue, blood, and everything else) or ‘fat free mass’. Preferably, dual energy X-ray absorptiometry, or DXA (formerly DEXA), is used as a method for estimating body fat percentage. Preferably, at least two different types of X-ray scans the body, one that detects all tissues and another that does not detect fat. A computer then subtracts the second picture from the first one, yielding only fat detection.

Body Average Density Measurement is another method of estimating body fat percentage by measuring a person's average density (total mass divided by total volume) and applying a formula to convert that to body fat percentage. The skilled artisan will recognize that this can be accomplished using the Siri and/or Brozek formulae. Body density can also be determined by hydrostatic weighing, which refers to measuring the apparent weight of a subject under water, with all air expelled from the lungs.

Alternatively, bioelectrical impedance analysis (BIA) is used to estimate body fat percentage. The general principle behind BIA is that two conductors are attached to a person's body and a small electrical charge is sent through the body. The resistance between the conductors will provide a measure of body fat, since the resistance to electricity varies between adipose, muscular and skeletal tissue.

Anthropometric methods for estimating body fat may also be used. The term anthropometric refers to measurements made of various parameters of the human body, such as circumferences of various body parts or thicknesses of skinfolds. Other formulas known to those of skill in the art for estimating body fat percentage from an individual's weight, girth and or height measurements, may also be used.

For example, an adipose tissue mass index can also be determined by calculating a body mass index (BMI). A BMI may be advantageous where the equipment and/or skill required to measure a body fat percentage is not readily available. Body mass index or Quetelet Index is a statistical measure of the weight of a person scaled according to height. BMI is defined as the individual's body weight divided by the square of their height. The formula preferably used produces a unit of measure of kg/m². A BMI may be used to assess how much an individual's body weight departs from what is normal or desirable for a person of his or her height. The weight excess or deficiency may, usually be accounted for by body fat (adipose tissue). Human bodies rank along the index from around 15 (near starvation) to over 40 (morbidly obese). This statistical spread is usually described in broad categories: underweight (about 15 to about 18), normal weight (about 19 to about 25), overweight (about 26 to about 30), obese (about 30 to about 40) and morbidly obese (about 40 and over).

An “essential” amino acid or “indispensable” amino acid is an amino acid that cannot be synthesized de novo by the organism, and therefore must be supplied in the diet. Nine amino acids are generally regarded as essential for humans. They are: isoleucine, leucine, lysine, threonine, tryptophan, methionine, histidine, valine and phenylalanine. Which amino acids are essential varies from species to species, as different metabolisms are able to synthesize different substances. For instance, taurine (which is not, by strict definition, an amino acid) is essential for cats, but not for dogs. Thus, dog food is not nutritionally sufficient for cats, and taurine is added to commercial cat food, but not to dog food.

The distinction between essential and non-essential amino acids is not strict, as some amino acids can be produced from others. For example, the sulfur-containing amino acids, methionine and homocysteine, can be converted into each other but neither can be synthesized de novo in humans. Likewise, cysteine can be made from homocysteine but cannot be synthesized on its own. So, for convenience, sulfur-containing amino acids are sometimes considered a single pool of nutritionally-equivalent amino acids. Likewise arginine, ornithine, and citrulline, which are interconvertible by the urea cycle, are considered a single group.

A diet “substantially deficient in an essential amino acid” can be any regimen of food over a particular time that is substantially deficient in one or more of any essential amino acid.

“Substantially deficient” means that a diet or dietary regimen has about 0 to about 20%, preferably about 0 to about 10%, more preferably about 0 to about 5% and most preferably less than 5% of the World Health Organization-recommended daily intake for human adults (mg per kg of body weight) of the essential amino acid.

Preferably, the substantially deficient essential amino acid is leucine. The World Health Organization-recommended daily intake for human adults for lecuine is 980 mg per 70 kg of body weight. One of skill in the art can readily determine the daily intake for human adults or children of different weights.

Preferably, the diet substantially free of leucine resembles the following 3-day regiment:

TABLE 2
SAMPLE MENUS FOR LOW LEUCINE DIET

Day 1
Breakfast
3 Pancakes (Low protein)
with maple syrup/margarine
Lunch
1 cup Cranberry-Apple Coleslaw
4 Vanilla Wafer Cookies (Low protein)
Dinner
1.5 Cups Veggie Stir-fry
1.5 Cups Low protein Macaroni
Snack 1 small Orange
Day 2
Breakfast
1 Cup Low protein Cereal Loops w/ Rice
Milk
⅓ Cup canned Grapefruit
Lunch
1.5 Cups Asian Crunch Salad
1 Cup Cranberry Juice
Dinner
1 Vegetarian Stuffed Pepper
Snack 10 Jelly Beans
Day 3
Breakfast
2 Apple-Cinnamon Muffins
⅓ Cup Fresh Strawberries
Lunch
Cucumber/Tomato Sandwich
Dinner
1 Cup Spaghetti with Tomato Sauce
Small Italian Salad
Snack Fresh Apple
All days include:
24 fl oz LMD low leucine drink
12 fl oz Xleu low leucine drink
2 tbsp. Xanthan gum for dietary fiber
* A normal leucine (NorLEU) group receives daily leucine supplement

TABLE 3
Leucine Free Diet Nutrients

| | | | | CHO | | fiber | leu |
|---|---|---|---|---|---|---|---|
| | serving | energy | fat g | g | pro g | g | mg |
| LMD low | 150 g | 750 | 39 | 76.5 | 24.3 | 0 | 0 |
| leucine drink | | | | | | | |
| powder | | | | | | | |
| Xleu low | 50 g | 153 | 0 | 17 | 20 | 0 | 0 |
| leucine drink | | | | | | | |
| powder | | | | | | | |
| Xanthan gum | 2 tbsp | 80 | 0 | 20 | 0 | 20 | |
| for dietary | | | | | | | |
| fiber** | | | | | | | |
| Average | | 1017 | 45.4 | 146.5 | 5.7 | | 180 |
| macronutrients | | | | | | | |
| from food | | | | | | | |
| Base diet | | 2000 | 84.4 | 260 | 50 | 20 | 180 |
| total | | | | | | | |
| | | | | | 38% | 52% | 10% |
| | | | | | fat | CHO | pro |

The term “General Control Nonderepressible 2 (GCN2) kinase” refers to a protein kinase that phosphorylates the alpha-subunit of translation initiation factor eIF2 in response to starvation. This activity is referred to as “GCN2 activity”.

Cells having a “GCN2 deficiency” refers to cells in which there less GCN2 activity compared to normal wild type cells. Preferably, in cells having a “GCN2 deficiency” the GCN2 activity is reduced, about 10 to about 50%, more preferably about 50 to about 75%, even more preferably about 75 to about 100% and most preferably, about 90 to about 100% or about 95 to about 100%.

The skilled artisan will recognize that a GCN2 deficiency in a cell can result through a multitude of mechanisms. For example, the cell can have a mutation in the GCN2 gene causing the complete or partial inhibition of the GCN2 gene transcription. Alternatively, the GCN2 gene can have a mutation that although not effecting the rate of GCN2 transcription nonetheless renders a mutant GCN2 mRNA that is not or not as efficiently translated as a wild type GCN2 mRNA. Alternatively, the GCN2 gene caries a mutation that results in a non- or only partially functional GCN2 protein.

Alternatively, a GCN2 deficiency in a cell is brought about through exogenous agents, i.e., “GCN2 antagonists”. For example, the skilled artisan may employ aptamers as GCN2 antagonists that bind a the endogenous GCN2 transcript or protein target.

In another example, small interfering RNA, sometimes known as short interfering RNA (siRNA), or silencing RNA, can be introduced to the cell to selectively knock down endogenous GCN2 expression. siRNA can be expressed by an appropriate vector, e.g. a plasmid. This is done by the introduction of a loop between the two strands, thus producing a single transcript, which can be processed into a functional siRNA.

A “GCN2 agonist” on the other hand, as the term is used herein refers to an agent or compound that mimics and/or increases GCN2 activity. For example, a GCN2 agonist can be a compound that increases GCN2 protein's ability or efficiency in phosphorylating the alpha-subunit of translation initiation factor eIF2. A GCN2 agonist can also be a compound that increases GCN2 expression. In an other example, a GCN2 agonist brings about the phosphorylation the alpha-subunit of translation initiation factor eIF2 in the absence of GCN2. In another example, a GCN2 agonist up regulates the expression of genes that are upregulated in a starvation response or down regulates the expression of genes that are down regulated in a starvation response. Given the present disclosure, the skilled artisan will recognize various additional ways in which a GCN2 agonist will mimic or increase the natural function of GCN2, particularly in a starvation response.

The GCN2 agonists envisaged by the disclosure are compounds projected to be effective in preferentially reducing adipose tissue in an individual. Compounds known to activate GCN2 are for example, but not limited to alcohol derivatives of some of the essential amino acids such as leucinol, histindol, and threoninol. Another example of a GCN2 agonist is the potent fatty acid synthase (FAS) gene expression inhibit C75.

Another example of a GCN2 agonist is a “tRNA aminoacylation inhibitor.” The inhibition of tRNA aminoacylation will activate an amino acid deprivation response resulting in up regulation of the activity of endogenous GCN2. The term as used herein relates to compounds and agents that inhibit the aminoacylation of tRNAs by their cognate aminoacyl tRNA synthetases. The aminoacylation of tRNAs attaches an amino acid to the 3′ end of a tRNA so that the amino acid can be delivered to the growing polypeptide chain as the anticodon sequence of the tRNA reads a codon triplet in a mRNA. The specificity of aminoacylation is determined by the ability of an aminoacyl tRNA synthetase (aaRS) to interact with the correct amino acid and to recognize its cognate tRNA through specific nucleotides in tRNA.

Aminoacyl-tRNA synthetases catalyze the esterification of a particular tRNA with its corresponding amino acid. In the first reaction step, the appropriate amino acid is recognized by the enzyme and reacts with ATP to form an enzyme-bound mixed anhydride; in the second step, this activated amino acid is esterified with one of the two hydroxyl groups of the tRNA. AaRSs are classified into two main groups of ten enzymes each, on the basis of common structural and functional features. Interference with either the amino acid binding step or the tRNA recognition step of a synthetase can inhibit aminoacylation and arrest protein synthesis.

tRNA aminoacylation inhibitors are compounds and agents that inhibit the aminoacylation of tRNAs by their cognate aminoacyl tRNA synthetases. As such, these compounds interference with either the amino acid binding step or the tRNA recognition step of a synthetase. Several amino acid analogs have proven useful as inhibitors of aminoacylation (see, e.g., Aldridge, K. E. (1992) Antimicrobial Agents and Chemotherapy 36, 851-853; Yanagisawa et al. (1994) J. Biol. Chem. 269, 24303-24309). See also U.S. Pat. No. 6,448,059).

Several natural products including pseudomonic acid, SB-203207, SB-219383, indolmycin, capsaicin and ascamycin are selective inhibitors of aminoacyl-tRNA synthetases. Pseudomonic acid is a potent inhibitor of bacterial IleRS and is an aminoacyl-tRNA synthetases inhibitor currently marketed as an antibacterial agent.

Synthetic inhibitors are preferably stable analogues of a mixed anhydride intermediate. The stability is preferably achieved by replacement of the labile anhydride function by non-hydrolyzable bioisosteres. Several aminoalkyl adenylates (replacement of the anhydride by a phosphate ester) and aminoacylsulfamoyl adenosines (replacement of the phosphate by a sulfamoyl group) have been synthesized and known by those of skill in the art to be potent inhibitors of aaRSs.

The critical role of GCN2 in regulating lipid metabolism was shown by the rapid development of liver steatosis in GCN2^{-/-} mice during leucine deprivation. Following a switch from a complete- to a leucine-deficient diet, GCN2^{+/+} mice down-regulate mRNAs encoding proteins linked to the synthesis of fatty acids and triglycerides including SREBP1c, ACL, FAS, SCD, G6PD, and ME. A previous in vitro study showed that FAS mRNA expression is repressed by leucine deprivation in HepG2 cells (Dudek and Semenkovich, 1995, J Biol Chem 270, 29323-29329.).

In contrast, GCN2^{-/-} mice fail to down-regulate these mRNAs, and some are even induced. Consistent with changes in its gene and protein expression, FAS enzyme activity is suppressed significantly by leucine deprivation in the livers of GCN2^{+/+} mice but increased 1.5-fold in GCN2^{-/-} mice. The importance of GCN2-dependent repression of FAS was further demonstrated by the ability of a potent FAS inhibitor to prevent liver steatosis in leucine-deprived GCN2^{-/-} mice. Increased fat accumulation in liver could also arise from other sources including mobilization of free fatty acids from adipose tissue and increased dietary intake (Fong et al., 2000, Hepatology 32, 3-10). However, leucine-deprived GCN2^{-/-} mice have a higher percentage of adipose tissue relative to body weight and consumed less food compared to mice fed a control diet. Thus, failure to repress lipogenesis is likely to be the major cause of liver steatosis in these mice.

The decreased expression of the lipogenic genes including FAS, ACL, SCD, G6PD and ME in response to leucine deprivation may reflect repression of transcription, inasmuch as these genes were previously shown to be regulated by transcription factor SREBP-1c as a function of nutritional state (Horton and Shimomura, 1999, Curr Opin Lipidol 10, 143-150). SREBP-1c belongs to a basic helix-loop-helix/leucine zipper transcription factor family, which also include SREBP-1a and SREBP2 (Brown and Goldstein, 1997, Cell 89, 331-340). Among the SREBP family of transcription factors, SREBP-1c preferentially regulates genes involved in triglyceride and fatty acid synthesis, whereas SREBP-2 regulates genes related to cholesterol synthesis. SREBP-1c mRNA and protein levels are reduced by leucine deprivation in the liver and this effect is GCN2 dependent. No change in the expression of SREBP-2 or its target genes were observed in the livers of either strain of mice under leucine deprivation PPARγ, a secondary activator of lipogenic gene expression in the liver (Herzig et al., 2003, Nature 426, 190-193), exhibits a modest reduction in the livers of leucine-deprived wild-type mice, and may therefore also contribute to the reduction in triglyceride synthesis.

In addition to altered triglycerides synthesis, impairment of β-oxidation, and/or imbalance between triglycerides uptake and secretion contributes to increased accumulation of triglycerides in the livers of GCN2^{-/-} mice. The transcription factor PPARα and its target genes related to β-oxidation are significantly upregulated in the livers of leucine-deprived GCN2^{-/-} mice. The increased expression of β-oxidation genes, however, is not sufficient to prevent the increased triglycerides accumulation in the livers of GCN2^{-/-} mice under these conditions. The increase of PPARα and its downstream targets in β-oxidation are a secondary response to the high level of triglyceride synthesis occurring in leucine-deprived GCN2^{-/-} mice.

In addition to its key role in regulating β-oxidation, PPARα has been shown to positively regulate the expression of genes controlling fatty acid uptake, including LPL, FABP, cd36 and FATP (Motojima et al, 1998, J Biol Chem 273, 16710-16714; Tordjman et al., 2001, J Clin Invest 107, 1025-1034). It was discovered that leucine deprivation results in upregulation of these PPARα target-genes facilitating fatty acid uptake into the liver. Thus, an increase in fatty acid uptake in leucine-deprived GCN2^{-/-} mice is likely a contributing factor to liver steatosis in these animals. However, expression of Apo B, which functions in the secretion of triglycerides from liver, was also upregulated by leucine deprivation, which would tend to reduce fatty acid accumulation. Moreover, further activation of PPARα with the agonist WY compound in mice deprived of leucine was not able to prevent the development of fatty liver in GCN2^{-/-} mice. Taken together, these results indicate that increases in fatty acid synthesis and uptake in the liver outweigh apparent increases in β-oxidation and secretion in GCN2^{-/-} mice, resulting in fatty liver.

In a starvation response, GCN2 causes the phosphorylation of the alpha-subunit of translation initiation factor eIF2 and also the down regulation of adipogenic genes, i.e., those associated with fat production and storage: fatty acid synthase (FAS), ATP-citrate lyase (ACL), stearoyl CoA desaturase (SCD), glucose-6-phosphate dehydrogenase (G6PD) and malic enzyme (ME), SREBP1c and SCAP.

It was also recognized that in a starvation response in an animal with a GCN2 deficiency, the following adipogenic genes are up regulated: fatty acid synthase (FAS), ATP-citrate lyase (ACL), glucose-6-phosphate dehydrogenase (G6PD), malic enzyme (ME), SREBP1c, SIP, PPAR-gamma, fatty aci-CoA oxidase (ACO), long and medium chain acyl-CoA dehydrogenase (LCAD and MCAD), fatty acid binding protein (FABP), fatty acid translocase (CD36), fatty acid transport protein (FATP), and lipoprotein lipase (LPL), alipoproteins B (ApoB). The skilled artisan will be able to readily identify other genes whose activity and/or gene products positively regulate fat production given the present disclosure and state of the art. Such genes are also adipogenic.

As such, another aspect of the disclosure provides for methods for identifying GCN2 agonists that will decrease the activity and/or expression of one or more of target genes. Such GCN2 agonists are envisaged to be useful agents for selectively inhibiting the cellular mechanisms that lead to fat production. Such agonists will also have the effect of reducing adipose tissue mass in an individual.

The preferred methods for identifying GCN2 agonists involve contacting a cell with a GCN2 deficiency with a compound suspected of being a GCN2 agonist and measuring whether the activity and/or expression of a particular target gene. The activity and/or expression of the particular gene is then compared to the activity and/or expression, respectively, of the same gene in the same cell type grown in the absence of the compound. The cells may be within an organism (in vivo) or in culture (in vitro).

Preferably, the cells are derived from a GCN2 deficient mouse. More preferably, the cells are derived from a GCN2 knockout mouse. However, as described above wild type cells may be treated with one or more GNC2 antagonists to bring about the GCN2 deficiency.

Preferably, the cells are fibroblasts, hepatocytes or any other preferably robust cell line that is readily grown in culture and amenable to experimental manipulation or standard techniques for assessing gene expression.

A target gene may be an adipogenic gene or a synthetic gene having the promoter region of an adipogenic gene operably linked to a reporter nucleic acid.

The term “promoter region” refers to a DNA sequence that functions to control the transcription of one or more nucleic acid sequences, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, calcium or cAMP responsive sites, and any other nucleotide sequences known to act directly or indirectly to regulate transcription from the promoter.

The term “operably linked” refers to the linkage of a DNA segment to another DNA segment in such a way as to allow the segments to function in their intended manners. A DNA sequence encoding a gene product is operably linked to a regulatory sequence when it is ligated to the regulatory sequence, such as, for example, promoters, enhancers and/or silencers, in a manner which allows modulation of transcription of the DNA sequence, directly or indirectly.

A “reporter nucleic acid sequence” is a DNA molecule that expresses a detectable gene product, which may be RNA or protein. The detection may be accomplished by any method known to one of skill in the art. For example, detection of mRNA expression may be accomplished by using Northern blot or RT-PCR analysis and detection of protein may be accomplished by staining with antibodies specific to the protein, e.g. Western blot analysis. Preferred reporter nucleic acid sequences are those that are readily detectable. Examples of reporter nucleic acid sequences include, but are not limited to, those coding for alkaline phosphatase, chloramphenicol acetyl transferase (CAT), luciferase, beta-galactosidase and alkaline phosphatase.

The expression and/or activity of the target gene can be measure at the level of mRNA transcription and/or translation and/or protein production and activity. For qualitatively or quantitatively measuring target gene transcription levels, preferred techniques include such assays as reverse transcriptase polymerase chain reaction (RT-PCR) or Northern blot techniques common in the are art.

Protein expression levels associated with a target genes are readily determined by standard SDS-PAGE, Western blot, ELISA or other well known techniques.

The preferred target genes to be utilized in a GCN2 deficient cellular background are the adipogenic genes: fatty acid synthase (FAS), ATP-citrate lyase (ACL), glucose-6-phosphate dehydrogenase (G6PD), malic enzyme (ME), SREBP1c, S1P, PPAR-gamma, fatty aci-CoA oxidase (ACO), long and medium chain acyl-CoA dehydrogenase (LCAD and MCAD), fatty acid binding protein (FABP), fatty acid translocase (CD36), fatty acid transport protein (FATP), insulin-induced gene-1 (Insig-1) and insulin-induced gene-2 (Insig-2), and lipoprotein lipase (LPL), alipoproteins B (ApoB).

One or more GCN2 agonists whether they be known or identified by the methods disclosed herein, are envisaged to be delivered to a patient or animal as a pharmaceutical composition with a pharmaceutically acceptable carrier; or as a dietary supplement.

The term “pharmaceutically acceptable carrier” includes a pharmaceutically acceptable material. A pharmaceutically acceptable carrier can include a single component or a composition and can be a liquid or a solid, such as a filler, diluent, excipient, solvent or encapsulating material. The pharmaceutical carrier is typically involved in carrying or transporting a compound(s) within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

A clinician, e.g., physician, veterinarian, or equivalent, having ordinary skill in the art can readily determine and prescribe the effective amount of the pharmaceutical composition required. For example, the physician or veterinarian could start doses of the compounds of the disclosure employed in the pharmaceutical composition, e.g., a GCN2 agonist, at levels lower than that required in order to achieve the desired therapeutic effect and gradually increase the dosage until the desired effect is achieved.

The methods, compounds, e.g., GCN2 agonists, and diets, e.g., those substantially deficient in an essential amino acid, envisaged by the present disclosure are useful for the treatment obesity, overweight and/or insulin resistance. However, the skilled artisan will also recognize that they are also suitable to treat conditions that are associated with overweight, obesity and/or insulin resistance. Such conditions are for example insulin resistance, diabetes and hypertension.

Around 30% of individuals who are at least 30 lbs. overweight have at least mildly elevated blood pressure. The etiology of this increase in blood pressure appears to be related to substances produced by adipose (fat) tissue and to the increase in the hormone insulin that occurs with obesity.

Obesity is also the leading cause of diabetes and insulin resistance. Type II diabetes is almost always associated with obesity and appears to be related to hormonal substances (cytokines) produced by adipose (fat) tissue and to the increase amount of blood lipids (fats) that occurs in diabetes. In the majority of obese individuals with diabetes, reducing body weight by 10% can eliminate or reduce the need for oral medications or insulin injections.

“Insulin resistance” is the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake, whereas insulin resistance in liver reduces glucose storage, with both effects serving to elevate blood glucose. High plasma levels of insulin and glucose due to insulin resistance often lead to metabolic syndrome and type 2 diabetes.

The “quantitative insulin sensitivity check index” (“QUICKI”) may be used to determine insulin resistance. It is derived using the inverse of the sum of the logarithms of the fasting insulin and fasting glucose: 1/(log(fasting insulin μU/mL)+log(fasting glucose mg/dL)).

This index correlates well with glucose clamp studies (r=0.78), and is useful for measuring “insulin sensitivity” (“IS”), which is the inverse of insulin resistance (“IR”). It has the advantage of that it can be obtained from a fasting blood sample, and is the preferred method for certain types of clinical research

Alternatively, a clinican can use the “glucose tolerance text” (“GTT”) to determine insulin resistance (or insulin sensitivity). A glucose tolerance test is the administration of glucose to determine how quickly it is cleared from the blood. The test is preferably used to test for diabetes, insulin resistance, and reactive hypoglycemia. The glucose is preferably given orally. The test may also be performed as part of a panel of tests, such as the comprehensive metabolic panel.

Preferably, the patient is instructed not to restrict carbohydrate intake in the days or weeks before the test. It is also preferable that the patient has fasted for the previous about 8 to about 14 hours, allowing only for water. Preferably, the GTT is scheduled to begin in the morning as glucose tolerance exhibits a diurnal rhythm with a significant decrease in the afternoon. A zero time (baseline) blood sample may then be drawn. The patient is then preferably given a glucose solution to drink. The standard dose is about 1.75 grams of glucose per kilogram of body weight, to about 75 g. Preferably, it is drunk within 5 minutes. Preferably, blood is drawn at intervals for measurement of glucose (blood sugar), and insulin levels. The skilled artisan will recognize that intervals and number of samples vary according to the purpose of the test. For example, in diabetes screening, the most important sample is the 2 hour sample and the 0 and 2 hour samples may be the only ones collected. In research settings, samples may be taken on many different time schedules. If renal glycosuria (sugar excreted in the urine despite normal levels in the blood), then urine samples may also be collected for testing along with the fasting and 2 hour blood tests.

Elevated cholesterol (hypercholesterolemia) is also commonly associated with obesity. On average, every 10 lbs of excess fat produces 10 mg of cholesterol per day. In other words, putting on 25 extra lbs. leads to the equivalent of taking in one extra egg yolk per day. Most people can successfully control their cholesterol by reducing both their fat intake and weight.

Other conditions associated with obesity include but are not limited to are stroke, hypothyroidism, dyslipidemia, hyperinsulinemia, glucose intolerance, congestive heart failure, angina pectoris, cholecystitis, cholelithiasis, osteoarthritis, gout, fatty liver disease, sleep apnea and other respiratory problems, polycystic ovary syndrome (PCOS), fertility complications, pregnancy complications, psychological disorders, uric acid nephrolithiasis (kidney stones), stress urinary incontinence, cancer of the kidney, endometrium, breast, colon and rectum, esophagus, prostate and gall bladder.

### EXAMPLE 1

### Leucine Deprivation Results in Radical Changes in Liver and Adipose Tissue Mass

GCN2^{+/+} and GCN2-mice were maintained on either a control diet or a diet lacking of leucine for 17 days. Leucine deprivation resulted in a reduction in food intake and body weight in both GCN2^{+/+} and GCN2^{-/-} mice (Table 1A). GCN2^{+/+} mice fed the leucine deprived diet for 17 days experienced a 48% loss of liver mass and a dramatic 97% loss of abdominal adipose mass, whereas GCN2^{-/-} mice showed no apparent loss of liver mass and much less severe loss of adipose tissue (69% reduced) compared to mice fed the control diet. A second leucine-deficient feeding experiment was conducted for a period of seven days (Table 1B) to better assess the more immediate effects of leucine-deprivation. Liver mass was similar in GCN2^{+/+} mice fed leucine-deprived diet compared to control diet during this shorter period, whereas adipose tissue was reduced by more than half. In contrast, liver mass was significantly increased in GCN2-deficient mice fed the leucine-deprived diet compared to complete diet, but no significant difference was seen in adipose mass.

### EXAMPLE 2

### GCN2^{-/-} Mice Display Severe Liver Steatosis Fed a Leucine-Deprived Diet

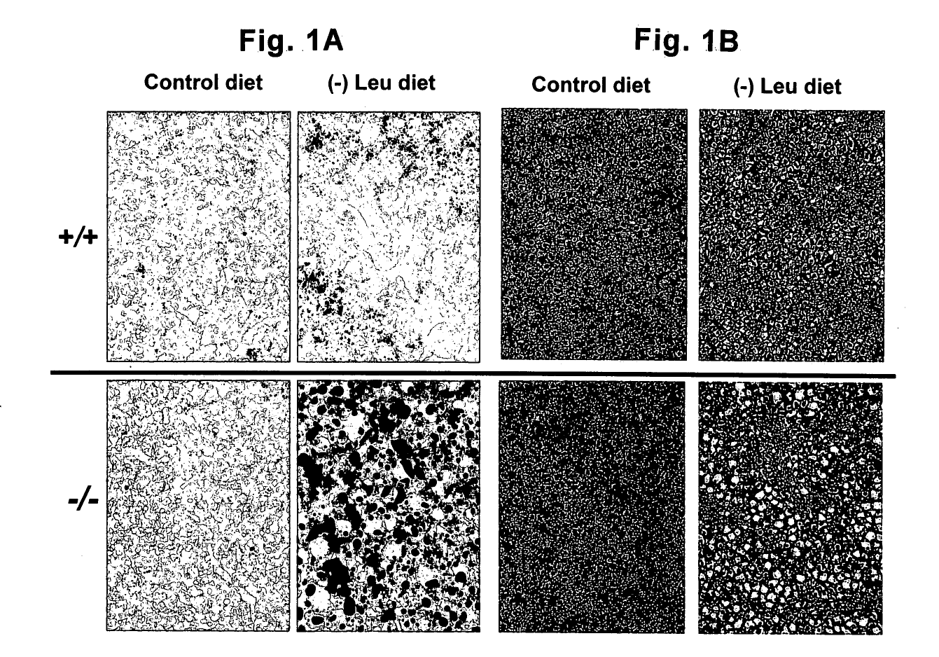

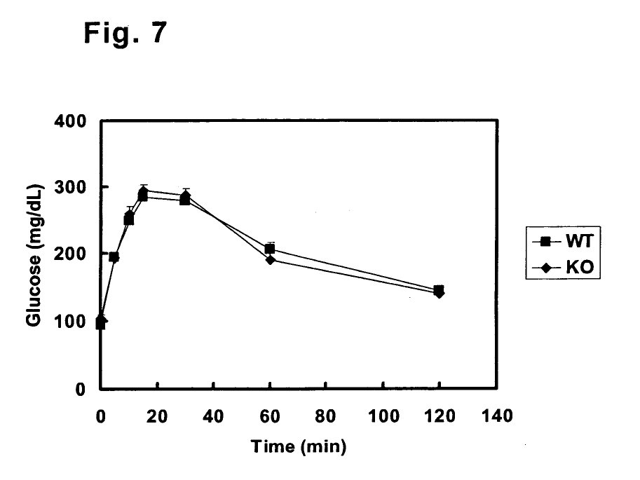

The livers of GCN2^{-/-} mice fed a leucine-deficient diet for 7 days appeared very pale, suggesting severe liver steatosis. The observation was confirmed by histological examination of the liver revealing extensive lipid deposition manifested as macro- and microvesicular steatosis (FIGS. 1A and B). Leucine deprivation had no effect on total cholesterol in the liver in either strain of mice. By contrast, leucine deprivation dramatically increased liver triglycerides in GCN2^{-/-} mice compared to GCN2^{+/+} mice (Table 1B). As observed for liver, cholesterol in serum was not affected by leucine deprivation in either GCN2^{+/+} or GCN2^{-/-} mice. Leucine deprivation significantly reduced triglycerides levels in serum in GCN2^{+/+} mice, and significantly more reduced in GCN2^{-/-} mice. Leucine deprivation resulted in significantly decreased free fatty acids in the serum in GCN2^{+/+} mice; in contrast, fatty acid levels increased in the serum in GCN2-mice. Taken together, these results indicate that leucine deprivation rapidly results in liver steatosis caused by deposition of triglycerides in the GCN2^{-/-} mice. Leucine deprivation resulted in decreased insulin levels in both genotypes perhaps in part due to decreased food intake, but serum glucose was unaffected by this diet (Table 1B). Liver glycogen content was equivalent among both diets and genotypes (Table 1B). Both strains of mice showed a normal glucose clearance when challenged to glucose injection (FIG. 7).

### EXAMPLE 3

### Genes Related to Triglyceride Synthesis are not Repressed in GCN2^{-/-} Mice Fed a Leucine-Deprived Diet

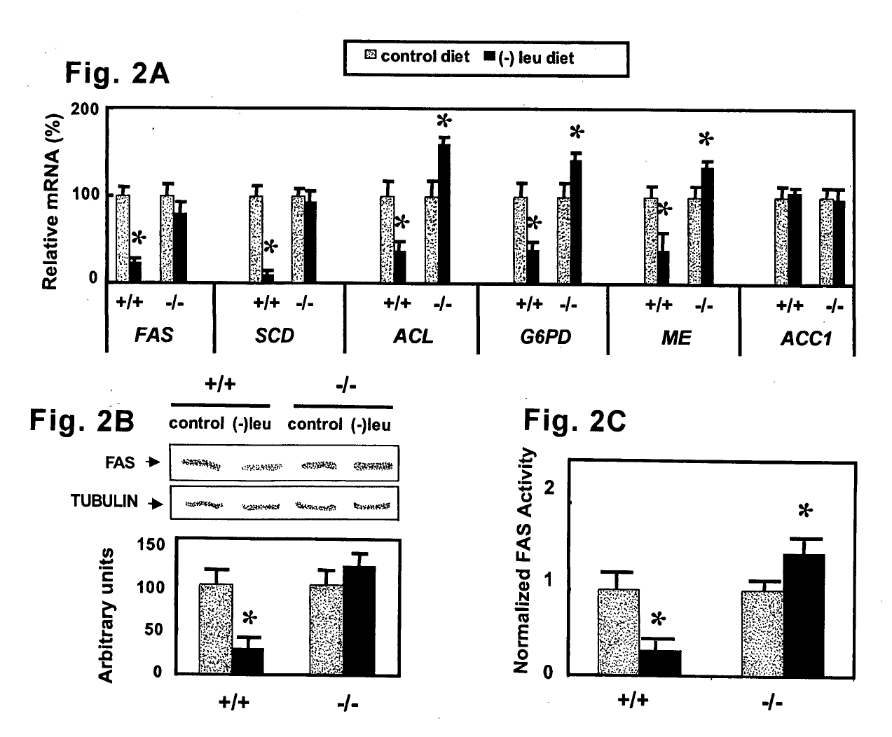

The accumulation of hepatic triglycerides in GCN2^{-/-} mice fed a leucine-deprived diet is likely to reflect an imbalance in hepatic triglyceride synthesis, β-oxidation, uptake and/or secretion of fatty acids. For this reason, levels of mRNA expression for proteins related to each of these processes were examined. It was first investigated whether genes underlying the synthesis of triglycerides are differentially regulated in GCN2^{+/+} and GCN2−/− mice during leucine deprivation. These proteins included acetyl CoA carboxylase (ACC)1 and fatty acid synthase (FAS), which together catalyzes rate-limiting step in production of palmitate 16:0; stearoyl CoA desaturase (SCD), which catalyses the synthesis of oleic acid (18:1, n−9); ATP-citrate lyase (ACL), which catalyzes the conversion of citrate to CoA and oxaloacetate, and glucose-6-phosphate dehydrogenase (G6PD) and malic enzyme (ME), which generate NADPH for synthesis of fatty acids (Horton and Shimomura, 1999, Curr Opin Lipidol 10, 143-150). Levels of FAS mRNA and protein were greatly reduced in the livers of GCN2^{+/+} mice fed on a leucine-deficient diet, but was not reduced in GCN2^{-/-} mice (FIGS. 2A and B). Similarly, FAS enzyme activity was reduced by 74% in the livers of GCN2^{+/+} mice, but was increased 1.5-fold in GCN2^{-/-} mice under leucine deprivation (FIG. 2C). Leucine deprivation also resulted in a large decrease in the mRNA levels of ACL, SCD, G6PD, and ME in the livers of GCN2^{+/+} mice. In contrast, the expression of these lipogenic genes was either not repressed (SCD) in the livers of leucine-deprived GCN2^{-/-} mice or in the case of ACL, G6PD and ME, their mRNA expression was increased (FIG. 2A). The increase in G6PD and ME expression may enhance the production of NADPH, which is required in copious amounts as reducing power to drive fatty acid synthesis. Only ACC mRNA, among the fatty acid synthesis genes, was expressed at equivalent levels in mice fed control or leucine-deficient diets in the livers of both GCN2^{+/+} or GCN2^{-/-} mice (FIG. 2A).

The expression of cholesterol synthesis genes including 3-hydroxy-3-methylglutaryl (HMG)-CoA synthase, HMG-CoA reductase and Farnesyl diphosphate synthase were unchanged in the livers of either GCN2^{+/+} or GCN2^{-/-} mice maintained on a leucine-deficient diet. These results are consistent with the observation that leucine deprivation did not result in a significant change in liver cholesterol levels in GCN2^{+/+} or GCN2^{-/-} mice (Table 1B).

### EXAMPLE 4

### Expression of SREBP-1c and PPARγ is Repressed in Wild Type but not GCN2^{-/-}-Mice, Fed a Leucine-Deprived Diet

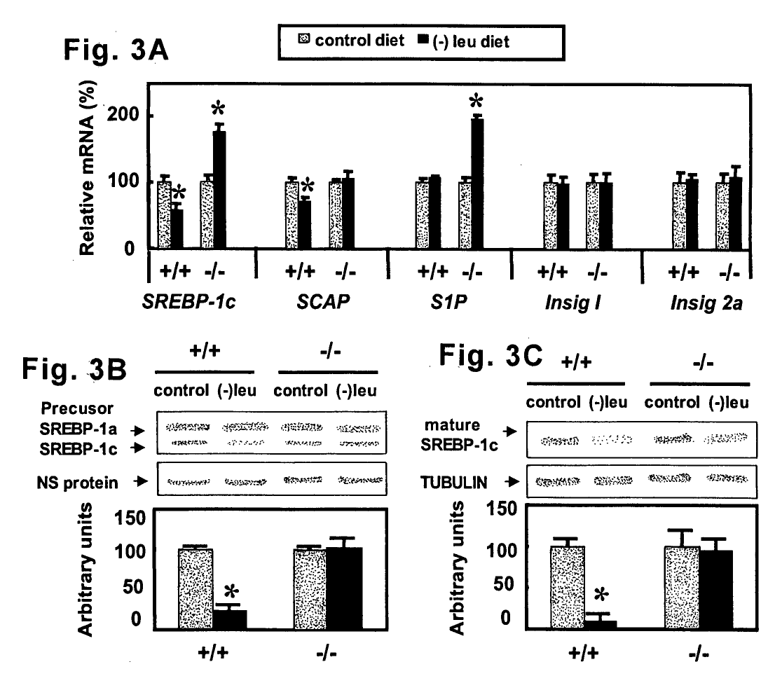

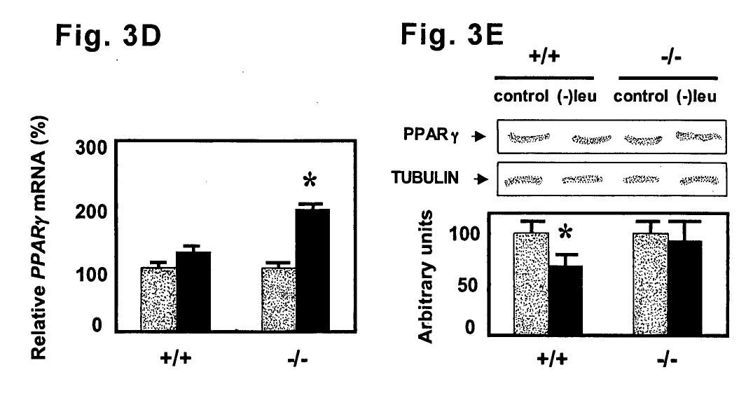

Lipogenic genes in the liver are predominately regulated by SREBP1c (Brown and Goldstein, 1997, Cell 89, 331-340) and secondarily by peroxisome proliferator-activated receptor (PPAR)γ (Herzig et al., 2003, Nature 426, 190-193) and carbohydrate response element binding protein (ChREBP) (Dentin et al., 2006, Diabetes 55, 2159-2170; Iizuka et al., 2006, Am J Physiol Endocrinol Metab 291, E358-364). Expression of SREBP-1c mRNA in liver was reduced 43% by leucine deprivation in GCN2^{+/+} mice, whereas it was induced by 176% in GCN2^{-/-} mice (FIG. 3A). Reduced expression of SREBP-1c mRNA in GCN2^{+/+} mice was accompanied by an 80% reduction in the precursor and mature, nuclear localized SREBP-1c protein, whereas it was unchanged in the livers of GCN2^{-/-} mice (FIGS. 3B and C). In contrast the SREBP-1a precursor protein was not altered in expression in either diet or genotype (FIG. 3B). Regulators of SREBP processing including SCAP, SIP, Insig 1 and 2a (Goldstein et al., 2006, Cell 124, 35-46) were examined. SCAP mRNA was reduced in the livers of GCN2^{+/+} mice and S1P mRNA was increased in the livers of GCN2^{-/-} mice under leucine deprivation, while the other genes remained unchanged in both strains of mice.

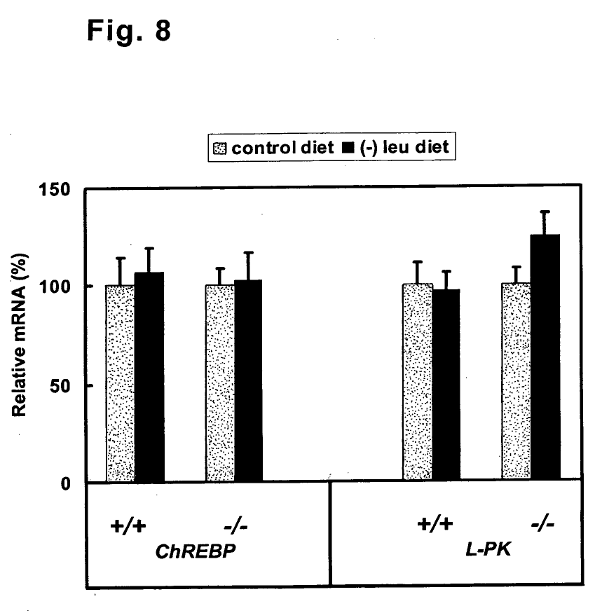

Leucine deprivation had no significant effect on levels of PPARγ mRNA in the livers of GCN2^{+/+} mice but PPARγ protein was moderately decreased (FIGS. 3D and E). Curiously, PPARγ mRNA was increased in the livers of leucine-deprived GCN2^{-/-} mice but PPARγ protein was unchanged (FIGS. 3D and E). Accumulation of fat could also be controlled by ChREBP-stimulated glycolysis and lipogenesis (Dentin et al., 2006, Diabetes 55, 2159-2170; Iizuka et al., 2006, Am J Physiol Endocrinol Metab 291, E358-364). Glucose levels, however, were unchanged in leucine-deprived GCN2^{+/+} or GCN2^{-/-} mice (Table 1B). The expression of ChREBP and its downstream target gene, Liver pyruvate kinase (L-PK), were also unchanged in the livers of either GCN2^{+/+} or GCN2-mice under leucine deprivation (FIG. 8).

### EXAMPLE 5

### β-Oxidation Genes are Unregulated in GCN2^{-/-} Mice Fed a Leucine-Deprived Diet

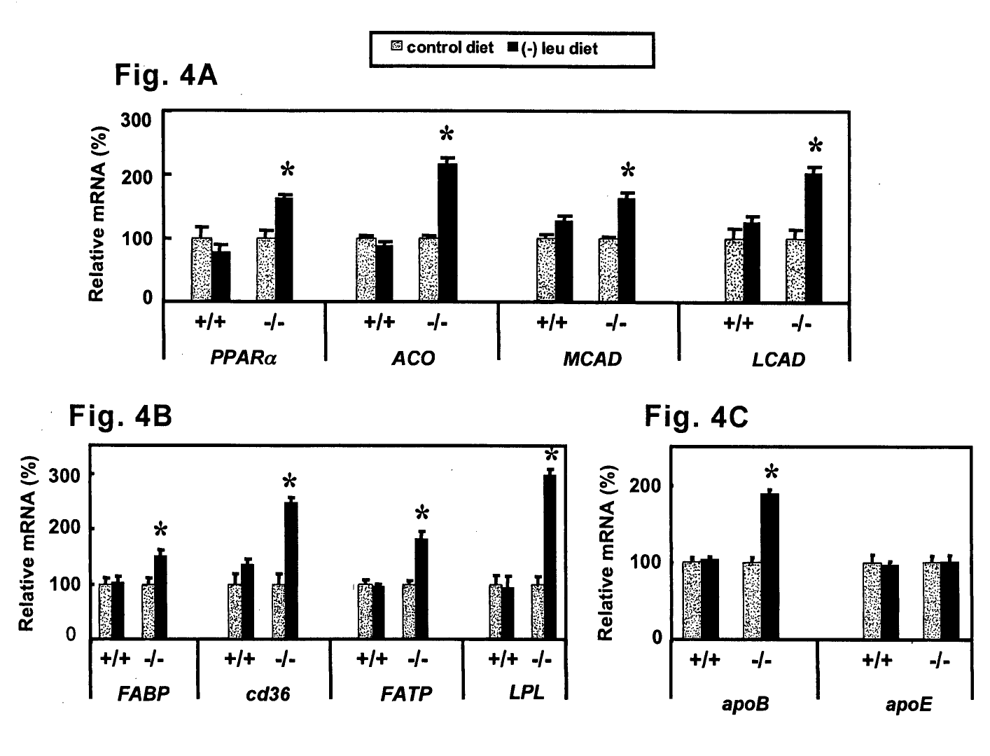

Impaired α-oxidation of fatty acids is another potential cause of liver steatosis (Kersten et al., 1999, J Clin Invest 103, 1489-1498; Leone et al., 1999, Proc Natl Acad Sci USA 96, 7473-7478). To examine if genes related to this process are differentially regulated by leucine deprivation in GCN2^{+/+} and GCN2^{-/-} mice, the expression level of mRNAs encoding transcription factor PPARα and its target genes fatty acyl-CoA oxidase (ACO), long and medium chain acyl-CoA dehydrogenase (LCAD and MCAD) were examined. Leucine deprivation had no effect on levels of PPARα, ACO, LCAD and MCAD mRNA in the livers of GCN2^{+/+} mice whereas the expression of these genes was increased markedly in GCN2-mice (FIG. 4A). Thus, the expression of β-oxidation genes is not impaired in GCN2^{-/-} mice but rather these genes are induced perhaps in response to increase fat accumulation in the liver.

### EXAMPLE 6

### Fatty Acid Transport Genes are Upregulated in GCN2^{-/-} Mice Fed a Leucine-Deprived Diet

In addition to triglyceride synthesis and β-oxidation, misregulation of triglyceride uptake and secretion could also contribute to fatty liver (Bradbury, 2006, Am J Physiol Gastrointest Liver Physiol 290, G194-198). Therefore mRNA from genes related to the uptake of fatty acid including fatty acid binding protein (FABP), fatty acid translocase (CD36), fatty acid transport protein (FATP) and lipoprotein lipase (LPL) was examined. These genes were all significantly increased in the livers of leucine-deprived GCN2^{-/-} mice whereas GCN2^{+/+} exhibit no significant increase in the expression. Expression of Apolipoproteins (Apo) B and E mRNAs, encoding key proteins in lipid secretion and transport, were also examined. ApoB mRNA was increased in the livers of leucine-deprived GCN2^{-/-} mice but Apo E mRNA was unchanged in both strains of mice (FIG. 4B).

### EXAMPLE 7

### Leucine Deprivation Induced-Liver Steatosis in GCN2^{-/-} Mice was Prevented by Treatment with C75, a Potent Fatty Acid Synthase Gene Expression Inhibitor

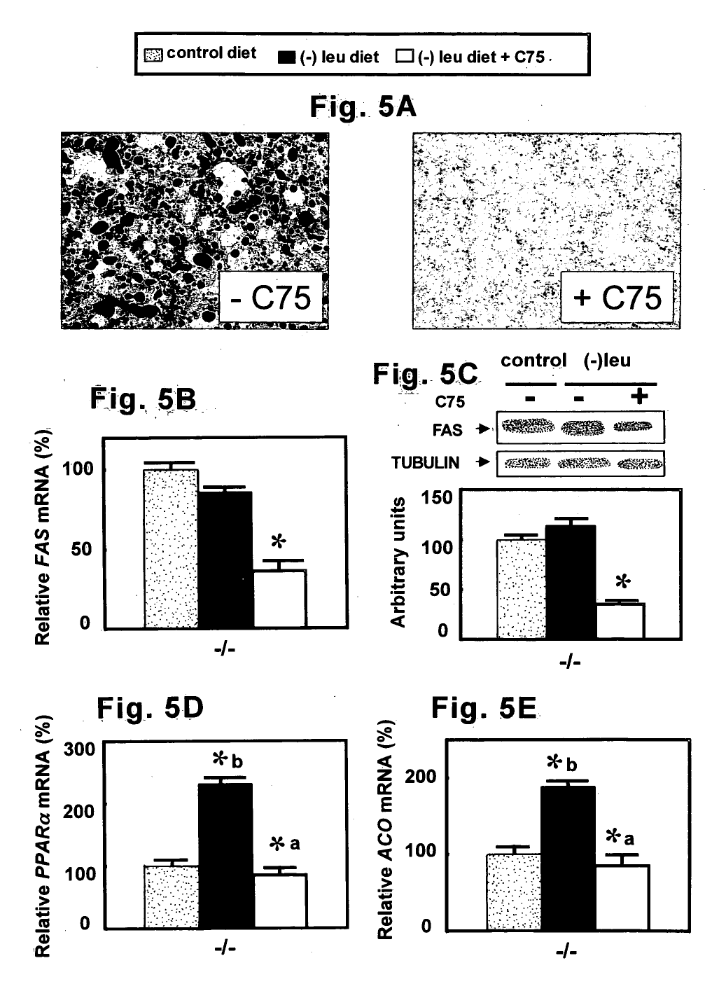

The failure to repress liver FAS expression and induced FAS enzyme activity were major factors contributing to persistent lipid synthesis and liver steatosis in leucine-deprived GCN2^{-/-} mice. The ability of C75, a potent FAS inhibitor, to repress lipid synthesis and the development of fatty liver in leucine-deprived GCN2^{-/-} mice was examined. C75 has been shown to inhibit FAS expression significantly in liver 24 hrs after a single IP injection (Kim et al., 2002, Am J Physiol Endocrinol Metab 283, E867-879). It was found that liver steatosis was prevented in leucine-deprived GCN2^{-/-} mice that had received three doses of C75 over the course of the seven-day period (FIG. 5A). Moreover, FAS mRNA and protein levels were repressed in the livers of GCN2^{-/-} mice (FIGS. 5B and C) to similar levels as seen in leucine-deprived wild-type mice (FIGS. 2A and B). The activation of PPARα mRNA and increased expression of β-oxidation genes in the livers of leucine-deprived GCN2^{-/-} mice was a secondary response to increased triglycerides and therefore should be ablated by C75 administration. Indeed, the levels of PPARα and ACO mRNA in the livers of leucine-deprived GCN2^{-/-} mice were reduced by C75 treatment (FIGS. 5D and E).

### EXAMPLE 8

### Phosphorylation of eIF2α and Regulation of Amino Acid Biosynthesis Genes are GCN2 Dependent in the Liver Under Leucine Deprivation

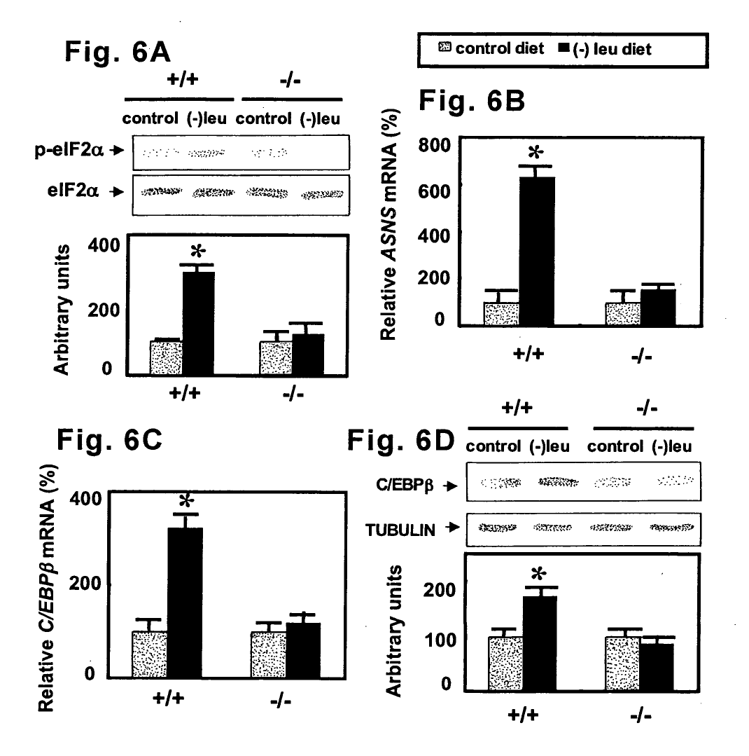

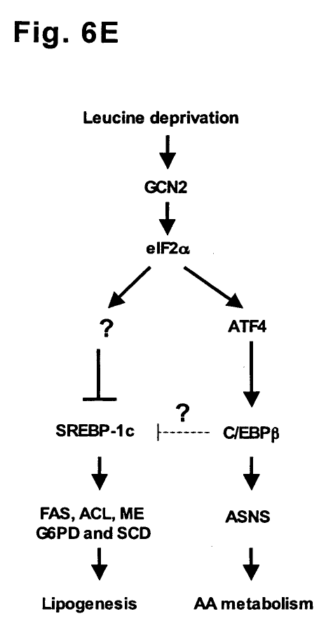

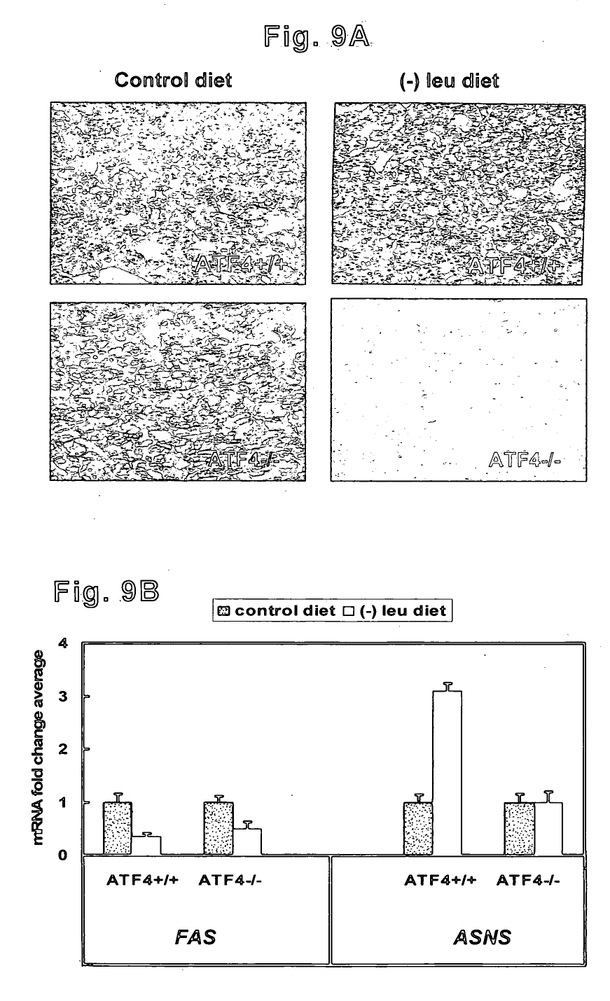

Short term leucine deprivation in cultured mouse embryonic fibroblasts was previously shown to result in potent activation of GCN2 and phosphorylation of eIF2α (Harding et al., 2003, Cell Metab 1, 273-277; Zhang et al., 2002b, Mol Cell Biol 22, 6681-6688). In response, ATF4 is induced translationally in cultured cells and activates the expression of C/EBPβ (Chen et al., 2005, Biochem J 391, 649-658) and amino acid metabolism and transport genes including asparagine synthetase (ASNS) (Harding et al., 2003, Mol Cell 11, 619-633; Siu et al., 2002, J Biol Chem 277, 24120-24127). However the GCN2-dependence of ASNS regulation had not been tested in a whole animal system. The phosphorylated state of eIF2α in the livers was markedly increased in wild-type mice consuming a leucine-deficient diet for seven days compared to mice fed complete diet, whereas GCN2^{-/-} mice lacked this induction (FIG. 6A). ASNS and C/EBPβ mRNA were expressed at low levels in the livers of both strains of mice when fed a complete diet (FIGS. 6B and C). The expression of these mRNAs, as well as the C/EBPβ protein, was induced markedly in the livers of leucine-deprived GCN2^{+/+} mice (FIG. 6 B-D). In contrast, ASNS mRNA and C/EBPβ mRNA and protein were not induced by the leucine-deprived diet in the livers of GCN2^{-/-} mice (FIG. 6 B-D). To determine if ATF4 was responsible for the GCN2-dependent regulation of lipogenesis and ASNS mRNA expression, ATF4^{-/-} mice were subjected to leucine-deprived diet for seven days. Unlike GCN2^{-/-} mice, ATF4^{-/-} mice did not develop fatty liver under leucine deprivation and liver FAS mRNA was repressed similar to ATF4^{+/+} mice (FIGS. 3A and B). However, ASNS mRNA was not induced in the livers of leucine-deprived ATF4-mice (FIG. 9) indicating that leucine deprivation-induced ASNS mRNA expression is dependent upon both GCN2 and ATF4 as was previously shown for cultured cells (Harding et al., 2003, Mol Cell 11, 619-633; Siu et al., 2002, J Biol Chem 277, 24120-24127).

### EXAMPLE 9

### Animals, Diets and C75 Treatment

Wild-type C57BL/6J (GCN2^{+/+}) mice were obtained from Jackson Laboratories (Bar Harbor, Me.). GCN2′-mice backcrossed onto the C57BL/6J background for eight generations were generated as previously described (Zhang et al., 2002, Mol Cell Biol 22, 6681-6688). All mice used were in C57BL/6J genetic background. 2- to 3-month-old male or female GCN2^{+/+} and GCN2^{-/-} mice were maintained on a 12-h light: dark cycle and provided free access to commercial rodent chow and tap water prior to the experiments. Control diet (a nutritionally complete amino acid) and (−) leu (leucine-deficient) diet were obtained from Research Diets, Inc. (New Brunswick, N.J.). All diets were isocaloric and compositionally the same in terms of carbohydrate and lipid component. At the start of the feeding experiment, mice were acclimated to control diet for 10 days, and then randomly assigned to either control diet group, continued free access to the nutritionally complete diet; or (−) leu diet group, free access to the diet that was devoid of the essential amino acid leucine for either 17 or 7 days. Experiments of treatment with C75 (Calbiochem), an inhibitor of fatty acid synthase gene expression, were performed in GCN2-mice fed on leucine-deficient diet. C75-treated group; mice were injected intraperitoneally (IP) with 20 mg/kg body weight C75 in 200 μl of RPMI at 2^{nd}, 4^{th} and 6^{th} day following on leucine-deficient diet; C75-untreated control group, mice were IP injected with 200 μl of RPMI accordingly. The dose of C75 was determined based on a previous report showing that 24 hrs after a single IP injection of C75 at a dose of 30 mg/kg body weight results in significant reductions of liver FAS expression (Kim et al., 2002, Methods Enzymol 71 Pt C, 79-85). Food intake and body weight were recorded daily. Animals were killed by CO₂ inhalation and trunk blood was collected for the assays described below. Body, liver and adipose tissue weight were recorded at the time of sacrifice. Livers were isolated and either put into 10% paraformaldehyde buffer right away for histological study or snap-frozen and stored at −20° C.

### EXAMPLE 10

### Serum Measurements

Serum was obtained by centrifugation of clotted blood and then snap-frozen in liquid nitrogen and stored at −20° C. Serum triglyceride, total cholesterol and free fatty acid levels were determined enzymatically using Triglycerol Reagent (Sigma), Infinity Cholesterol Reagent (Thermo) and NEFA C Reagent (Wako), respectively. Serum insulin was measured using Mercodia Ultrasensitive Rat Insulin ELISA kit (ALPCO Diagnostic).

### EXAMPLE 11

### Liver Lipid and Glycogen Assays

Lipids were extracted using chloroform/methanol (2:1, v/v) and evaporated in heat block and the pellets were dissolved in water as previously described (Herzig et al., 2003, Nature 426, 190-193). Liver triacylglycerol and total cholesterol contents were determined using the commercial reagent or kits described above. Values were calculated as mg per g wet tissue. Liver glycogen content was assayed using glucose assay reagent (G3293, sigma) using enzyme amyloglucosidase (sigma) as previously described (Passonneau and Lauderdale, 1974, Anal Biochem 60, 405-412). Values were calculated as μmol/mg wet tissue.

### EXAMPLE 12

### Oil Red O and Hematoxylin and Eosin

Frozen sections of the liver (5 μm) ware stained with Oil Red O. Formalin-fixed, paraffin-embedded sections (5 μm) of liver were stained with hematoxylin and eosin for histology.

### EXAMPLE 13

### FAS Enzyme Activity Assay

FAS activity was performed as described previously (Kim et al., 1981, Methods Enzymol 71 Pt C, 79-85) with some modifications. The rate of NADP(H) oxidation was measured at 340 nm before and after addition of the substrate malonyl-CoA. The concentration of enzyme was adjusted to assure a linear reaction rate.

### EXAMPLE 14

### RNA Isolation and Relative Quantitative RT-PCR

Total RNA was prepared from frozen liver using an RNase mini kit (Qiagen). Quantitative RT-PCR was performed using co-amplification of GAPDH as an internal control. 1 μg of RNA was reverse transcribed with random primers (Promega) and then quantitative PCR was performed with the qPCR core kit for sybergreen I (Eurogentec) by 7000 sequence detection system (Applied Biosystems). PCR products were subjected to a melting curve analysis. At a specific threshold in the linear amplification stage, the cycle differences between amplified GAPDH and cDNA of interest were used to determine the relative quantity of genes of interest.

### EXAMPLE 15

### Western Blot Analysis

Nuclear extraction from frozen liver was performed as previously described (Tai et al., 2000). Whole cell lysate from frozen liver were isolated using Tris-based lysis buffer. Protease inhibitor and phosphatase inhibitors were added to all buffers before experiments. Western blot was performed as previously described (Zhang et al., 2002, Mol Cell Biol 22, 6681-6688) using 30 μg protein for each sample. Protein concentration was assayed using Bio-Rad reagent. Primary antibodies anti-FAS antibody (BD scientific), anti-PPARγ antibody (Upstate) and anti-SREBP-1c antibody (Santa Cruz Biotechnology) were incubated overnight at 4° C. followed by ECL Plus (Amersham/Pharmacia) visualization of specific proteins. Band intensities were measured using ImageQuant (Molecular Dynamics/Amersham/Pharmacia). Intensity values were normalized to α-tubulin or non-specific protein on the membrane.

### EXAMPLE 16

### Data Analyses

All data are expressed as mean ±SEM for the experiments included numbers of mice as indicated. The two-tailed student t-test was used to evaluate statistical differences between control diet group and (−) leu diet group with or without C75 treatment within the same strains of mice. There was no significant effect of mouse strain on parameters shown when fed on control diet. Means for all parameters examined in current study were calculated independently for male and female mice. No statistical differences in the response of male and female mice to leucine deprivation was observed (two-tailed student t-test, p>0.05).

### EXAMPLE 17

### Increased Insulin Sensitivity

A reduction in circulating insulin in combination with stable and normal glucose homeostasis, suggests an increase in insulin sensitivity. Previously, Anthony, et al. 2000, and Anthony et al. 2004, showed reduced serum insulin levels in mice deprived of leucine. However, in those studies, serum insulin levels were not measured in fasting mice. Moreover, glucose levels were not assayed. As such, these studies did not suggest that a leucine free diet had any actual impact on insulin sensitivity.

Alternatively, it was found herein, that mice deprived of leucine exhibit a substantial reduction in serum insulin without a significant change in serum glucose levels. The QUICKI index is an estimator of potential insulin sensitivity that is based upon two parameters: serum insulin and serum glucose levels. Katz et al., J Clin Endocrinol Metab. 2000 July; 85(7):2402-10. Using the QUICKI index, it was estimated that the insulin sensitivity was increased from 0.48 to 0.65 in the leucine-deficient diet compared to the complete diet after one week. Normally, QUICKI index is an accurate estimator of insulin sensitivity when insulin and glucose have been determined after the required period of fasting (i.e. fasting glucose and insulin determinations) (Katz, 2000). QUICKI index results are not accurate with out the requisite fasting period.

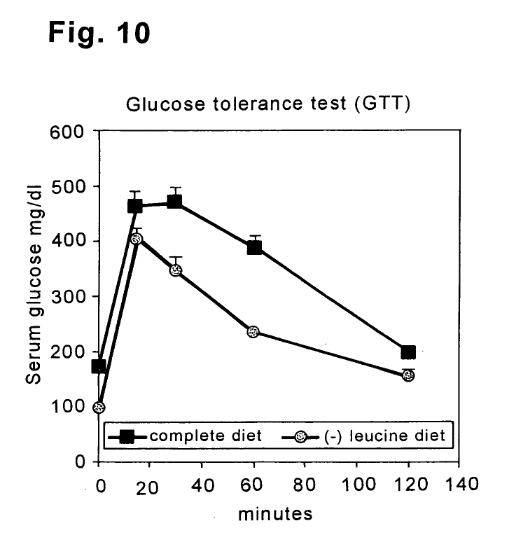

In order to confirm the QUICKI index test results, glucose tolerance tests (GTT) were performed on mice which had been subjected to a leucine-deficient diet for seven days compared to control animals which had been on a synthetic complete diet. It was found that leucine-deprived mice showed a substantial improvement in glucose tolerance (FIG. 10) compared to the control mice.

### EXAMPLE 18

### Human Low Leucine Diet Experiment

A low leucine diet (LowLEU) experiment will be conducted over 14 days and will entail 48 test subjects half of which will be administered the LowLEU diet (7% recommended daily amount or “RDA”) and half of which will be administered an identical diet but with normal leucine levels. The subjects will not be aware of which of the two diets they are being given, and they will be asked to record their opinion on the palatability of the diet. Body composition and metabolic parameters will be assessed before, during and after the 14-day treatment period. Test diet: LowLEU 7% Leucine—combined with 25% caloric restriction; Control diet: NormLEU 100% Leucine—combined with 25% caloric restriction.

Test and control diets will be identical in composition and amount with the exception of purified leucine added to the formula component in the control diet to restore normal leucine levels. The diets will be composed of a combination of synthetic complete nutritional formulas that are specifically missing leucine and foods containing no protein or very little protein (see proposed diets in Tables 2 and 3). The formulas to be used include LMD® (Mead Johnson) and XLeu® (Nutricia), which are currently used for treatment of isovalaric acidemia and are safe for human consumption under the supervision of a physician or nutrionist.

A moderate level of caloric restriction (25%) will also be imposed because in rodent studies animals reduce caloric intake when fed a leucine-deficient diet and it is desirable to mimic the rodent studies. In order to determine the appropriate caloric restriction for each subject in this study, the resting metabolic rate (RMR) will be measured before the beginning of the diet period to estimate the daily calorie intake necessary to maintain current body weight. This value, with an added activity factor, will be multiplied by 0.75 to achieve a 25% caloric restriction.

The LowLEU diet or a normal leucine diet will be administered for 14 days to overweight subjects (BMI=25-30). The two diet groups will each consist of 24 subjects, 12 male and 12 female adults 30-60 years old. The subjects will be using the inclusion and exclusion criteria listed below. The study will be conducted as single-blind experiments and therefore the subjects will not know the identity of their diet (LowLeu or NormLeu).

The 48 subjects (24 men, 24 women) will be assigned to either the low leucine (LowLEU) diet or the normal leucine (NormLEU) diet using an age stratified assignment design for each gender (see Data Analysis). The diet period will be 14 days. The diet will consist of a three-day cycle menu with three meals and one snack daily. Within the constraints of protein restriction, macronutrient content will conform as closely as possible to Dietary Reference Intakes (DRIs) acceptable distributions: 10% protein, 35% fat, and 55% carbohydrate, with 20 grams of dietary fiber daily. The LowLEU diet will contain 7% of the RDA for leucine and the NorLEU diet will provide 100% of the RDA for leucine, based on a mean of the figures for adult men and women of average height and weight. All foods will be weighed to the nearest tenth of a gram.

The two diets will consist of exactly the same foods and nutrients except for the leucine content. To achieve this, the LowLEU group will be given leucine free beverages made from special dietary products manufactured for people with such disorders as isovaleric academia that cannot metabolize leucine (LMD leucine-free diet powder, Mead Johnson, and XLeu Maxamum, Nutricia North America). For the NorLEU group, leucine supplements will be added to achieve the RDA for leucine without changing any other component of the diet. Because leucine is the most abundant amino acid in nature, only very small amounts of food can be included. To add variety, special commercial low protein foods will be used due to their low leucine content (Low proteinfin Foods, United Kingdom). Due to the small quantities and types of foods that can be calculated into the diet, dietary fiber will come mainly from xanthan gum. Xanthan gum is a cellulose-based product used as a thickener and stabilizer. It contains 10 grams of dietary fiber per tablespoon. See Table 2, supra.

A base diet of 2000 kilocalories will be calculated. Using each individual's measured resting metabolic rate (RMR) and appropriate activity factor, a 25% deficit in total kilocalorie needs will be determined for daily intake to affect a gradual weight loss of 2-5 lb over the 2-week test period. Proposed menus are shown in Table 2 and nutrient breakdown is shown in Table 3.

Blood samples will be drawn three times for each subject (before and after the diet experiment and after one week during the two week diet period). Serum will be used for a number of assays and measurements (listed below). The purpose of these assays are two fold: (1) assess and monitor health of subjects to assure safety of the diet and (2) assess the metabolic consequences of the diet including insulin sensitivity, release of adipokines from adipose tissue, and levels of fatty acids, triglycerides, cholesterol, etc. that may be impacted by the loss of adipose tissue.

Assays will include: General health analysis including EKG; Resting metabolic rate (RMR) (before to determine appropriate caloric intake to achieve 25% caloric restriction); Body mass index (BMI) (before, 1 week, and after diet); Body composition analysis by DEXA (before and after diet).

Serum assays after one week, and at the end of the 14-day diet period will include measuring: glucose, insulin, QUICKI index (insulin sensitivity from fasting glucose and insulin data), FFA (free fatty acids), triglycerides, cholesterol, HDL, LDL, Ketones, AST (liver function), ALT (liver function), Albumin (liver function), BUN− blood urea nitrogen (kidney function), Creatinine (kidney function), Creatine kinase CKMM/CPK-3 (skeletal muscle break down), Creatine kinase CKMB (heart muscle break down), CBC, Bilirubin.

The following adipokines will also be assayed immediately before and after diet period: Adiponectin, PAI-1 active, Resistin, IL-1b, Il-6, IL-8, Insulin, Leptin, MCP-1, and TNFα.

Several biomarkers of metabolism and assessment of body composition will be performed. Key among these markers is serum insulin, which is predicted to show a significant decrease in the LowLeu group compared to the NormLeu group. The average serum insulin level is 17.0 ng/ml for adult females and 21.2 ng/ml for adult males less than 60 years old with a coefficient of variation (CV) 8.3% within each sex. In addition to serum insulin, other variables including the components of body composition will be measured. The population variance for body composition is substantially less than observed for serum insulin, and therefore we chose to determine the number of subjects required for this study based upon serum insulin, which should therefore provide sufficient replication for other variables. It is evisaged that a 10% (ca.+/−2 ng/ml) difference in the mean serum insulin level between these two groups at the end of the diet study will be determined. Therefore the number of subjects required for each treatment in the proposed study was determined to be 12 by performing a power test (power=0.8, a=0.05) to determine a 10% mean difference in serum insulin levels (CV=8.3%). The mean difference in serum insulin levels will be determined independently for each gender. Therefore the proposed study will include 24 adult males and 24 adult females divided evenly into two groups: normal leucine (NormLeu) and low-leucine (LowLeu). Thus, each diet group will contain 12 adult males and 12 adult females. To achieve equivalent groups, subjects will be stratified randomly based upon age (see Screening Subjects above). Given the brief duration of the diet study we anticipate a small drop-out rate of <10%. Thus to achieve a sample size of 12 individuals per group, we expect that 1-2 individuals per group will need to be added over the course of the study.

The data will first be tested to determine if the parametric assumptions are met (i.e. normal distribution and equivalent variances) prior to subjecting the data to parametric statistical analysis. Analysis of variance (ANOVA) and other appropriate parametric statistical analysis (e.g. student t test) will be performed to test for statistical differences within and between groups (treatment and gender). Non-parametric tests such as the Mann-Whitney-Wilcoxon test, will also be performed.

In this disclosure there is described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention. All references cited herein are incorporated by reference in their entirety for all purposes.

What is claimed is:

1. A method of preferentially reducing adipose tissue mass in an animal in need thereof comprising, providing the animal with a diet substantially deficient in an essential amino acid for a period of time.

2. The method of claim 1 wherein the animal is a human.

3. The method of claim 1 where the method further comprises observing a reduction of adipose tissue mass by measuring a reduction in the adipose tissue mass index.

4. The method of claim 3 wherein the adipose tissue mass index is measured using a technique selected from the group consisting of bioelectrical impedance analysis, dual energy X-ray absorptiometry and determination of a body mass index (BMI).

5. The method of claim 1 wherein the essential amino acid is leucine.

6. The method of claim 1 wherein the period of time is less than 1 year.

7. The method of claim 1 wherein the period of time is less than 6 months.

8. The method of claim 1 wherein the period of time is less than 3 months.

9. The method of claim 1 wherein the period of time is less than 1 month.

10. The method of claim 1 wherein the period of time is less than 2 weeks.

11. The method of claim 1 wherein the period of time is less than 1 week.

12. A method of reducing adipose tissue mass index in an animal in need thereof comprising providing the animal with a diet comprising a GCN2 agonist for a period of time; and optionally, observing a reduction in adipose tissue mass index.

13. The method of claim 12 wherein the animal is a human.

14. The method of claim 12 wherein the GCN2 agonist is selected from the group consisting of leucinol, histindol, and threoninol.

15. The method of claim 12 wherein the GCN2 agonist is a tRNA aminoacylation inhibitor.

16. The method of claim 15 wherein the inhibitor of tRNA aminoacylation is selected from the group consisting of pseudomonic acid, SB-203207, SB-219383, indolmycin, capsaicin and ascamycin, an aminoalkyl adenylate and an aminoacylsulfamoyl adenosine.

17. The method of claim 12 wherein the period of time is less than 1 year.

18. The method of claim 12 wherein the period of time is less than 6 months.

19. The method of claim 12 wherein the period of time is less than 3 months.

20. The method of claim 12 wherein the period of time is less than 1 month.

21. The method of claim 12 wherein the period of time is less than 2 weeks.

22. A method of identifying a GCN2 agonist:
a) Growing a test cell with a GCN2 deficiency in an environment substantially free of an essential amino acid;
b) Growing a control cell with a GCN2 deficiency in the environment;
c) Contacting the test cell a compound suspected of being a GCN2 agonist;
d) Identifying a GCN2 agonist where the test cell expresses a lower level of a target gene than the control cell.

23. The method of claim 22 wherein the GCN2 deficiency is the result of a mutation in the cell's GCN2 gene.

24. The method of claim 22 wherein the GCN2 deficiency is the result of an GCN2 antagonist.

25. The method of claim 22 wherein the essential amino acid is leucine.

26. The method of claim 22 where in the expression is measured by determined by measuring target gene mRNA levels.

27. The method of claim 22 wherein the expression level is determined by measuring target gene protein product levels.

28. The method of claim 22 wherein the target gene is an adipogenic gene.

29. The method of claim 22 wherein the target gene comprises the promoter region of an adipogenic promoter region operably linked to a reporter nucleic acid.

30. The method of claim 28 wherein the adipogenic gene is selected from the group consisting of fatty acid synthase (FAS), ATP-citrate lyase (ACL), glucose-6-phosphate dehydrogenase (G6PD), malic enzyme (ME), SREBP1c, S1P, PPAR-gamma, fatty aci-CoA oxidase (ACO), long and medium chain acyl-CoA dehydrogenase (LCAD and MCAD), fatty acid binding protein (FABP), fatty acid translocase (CD36), fatty acid transport protein (FATP), and lipoprotein lipase (LPL), alipoproteins B (ApoB).

31. A method of increasing insulin sensitivity in an animal in need thereof comprising, providing the animal with a diet substantially deficient in an essential amino acid for a period of time.

32. The method of claim 31 wherein the animal is a human.

33. The method of claim 31 where the method further comprises observing an increase in insulin sensitivity by measuring glucose and insulin levels in the animal.

34. The method of claim 33 wherein the increase in insulin sensitivity is measured using a quantitative insulin sensitivity check index (QUICKI) or a glucose tolerance test (GTT).

35. The method of claim 31 wherein the essential amino acid is leucine.

36. The method of claim 31 wherein the period of time is less than 1 year.

37. The method of claim 31 wherein the period of time is less than 6 months.

38. The method of claim 31 wherein the period of time is less than 3 months.

39. The method of claim 31 wherein the period of time is less than 1 month.

40. The method of claim 31 wherein the period of time is less than 2 weeks.

41. The method of claim 31 wherein the period of time is less than 1 week.

42. A method of increasing insulin sensitivity in an animal in need thereof comprising providing the animal with a diet comprising a GCN2 agonist for a period of time; and optionally, observing an increase in insulin sensitivity.

43. The method of claim 42 wherein the animal is a human.

44. The method of claim 42 wherein the GCN2 agonist is selected from the group consisting of leucinol, histindol, and threoninol.

45. The method of claim 42 wherein the GCN2 agonist is a tRNA aminoacylation inhibitor.

46. The method of claim 45 wherein the inhibitor of tRNA aminoacylation is selected from the group consisting of pseudomonic acid, SB-203207, SB-219383, indolmycin, capsaicin and ascamycin, an aminoalkyl adenylate and an aminoacylsulfamoyl adenosine.

47. The method of claim 42 wherein the period of time is less than 1 year.

48. The method of claim 42 wherein the period of time is less than 6 months.

49. The method of claim 42 wherein the period of time is less than 3 months.

50. The method of claim 42 wherein the period of time is less than 1 month.

51. The method of claim 42 wherein the period of time is less than 2 weeks.
